(19) United States
(12) Patent Application Publication
(10) Pub. No.: US 2024/0270719 A1
(43) Pub. Date: Aug. 15, 2024

(54) CRYSTALLINE FORMS OF 2-(4-(4-(AMINOMETHYL)-1-OXO-1,2-DIHYDROPHTHALAZIN-6-YL)-1-METHYL-1H-PYRAZOL-5-YL)-4-CHLORO-6-CYCLOPROPOXY-3-FLUOROBENZONITRILE

(71) Applicant: Mirati Therapeutics, Inc., San Diego, CA (US)

(72) Inventors: Michal Achmatowicz, San Diego, CA (US); Svitlana Kulyk, San Diego, CA (US); Christopher Ronald Smith, San Diego, CA (US); Nan Wang, South Plainfield, NJ (US); Harsh Shah, South Plainfield, NJ (US); Tian Xie, South Plainfield, NJ (US); Qi Gao, South Plainfield, NJ (US)

(21) Appl. No.: 18/437,950

(22) Filed: Feb. 9, 2024

Related U.S. Application Data
(60) Provisional application No. 63/484,599, filed on Feb. 13, 2023.
(60) Provisional application No. 63/516,673, filed on Jul. 31, 2023.

(51) Int. Cl. C07D 403/04

(52) U.S. Cl. CPC C07D 403/04

(57) ABSTRACT

Disclosed herein are crystalline forms of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile free base, pharmaceutically acceptable compositions comprising these crystalline forms, and methods for using these crystalline forms.

### CROSS-REFERENCE TO RELATED APPLICATIONS

This application claims priority from U.S. Provisional Application No. 63/484,599, filed Feb. 13, 2023, and U.S. Provisional Application No. 63/516,673, filed Jul. 31, 2023, the disclosure of each of which is hereby incorporated by reference in its entirety.

### FIELD OF THE INVENTION

This invention relates to free bases of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile and particular crystalline forms thereof, processes of preparing the crystalline forms, and pharmaceutical compositions including the crystalline forms. The crystalline forms thereof are useful in the treatment and/or prevention of diseases and/or conditions related to cell proliferation, such as cancer. In particular, the crystalline forms provide therapeutic benefits as MTA-cooperative inhibitors of Protein Arginine N-Methyl Transferase 5 (PRMT5).

### BACKGROUND OF THE INVENTION

Protein Arginine N-Methyl Transferase (PRMT5) is a type II arginine methyltransferase that catalyzes the transfer of a methyl group from S-adenosyl-L-methionine (SAM) to an omega-nitrogen of the guanidino function of protein L-arginine residues (omega-monomethylation) and the transfer of a second methyl group to the other omega-nitrogen, yielding symmetric dimethylarginine (sDMA). PRMT5 forms a complex with MEP50 (methylosome protein 50), which is required for substrate recognition and orientation and is also required for PRMT5-catalyzed histone 2A and histone 4 methyltransferase activity (e.g., see Ho et al., (2013) PLOS ONE 8(8): 10.1371/annotation/e6b5348e-9052-44ab-8f06-90d01dc88fc2).

Homozygous deletions of p16/CDKN2a are prevalent in cancer and these mutations commonly involve the co-deletion of adjacent genes, including the gene encoding methylthioadenosine phosphorylase (MTAP). It is estimated that approximately 15% of all human cancers have a homozygous deletion of the MTAP gene (e.g., see Firestone & Schramm (2017) J. Am. Chem Soc. 139(39):13754-13760. doi: 10.1021/jacs.7b05803. Epub 2017 September 20).

Cells lacking MTAP activity have elevated levels of the MTAP substrate, methylthioadenosine (MTA), which is a potent inhibitor of PRMT5. Inhibition of PRMT5 activity results in reduced methylation activity and increased sensitivity of cellular proliferation to PRMT5 depletion or loss of activity. Hence, the loss of MTAP activity reduces methylation activity of PRMT5 making the cells selectively dependent on PRMT5 activity.

Thus, MTA-cooperative inhibition of PRMT5 activity in MTAP deleted cancers can provide therapeutic benefit for a wide range of cancers. The compounds of the invention provide this therapeutic benefit as MTA-cooperative inhibitors of PRMT5 that negatively modulate the activity of MTA-bound PRMT5 in a cell, particularly an MTAP-deficient cell, or for treating various forms of MTAP-associated cancer.

In particular, 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile, is a potent and selective inhibitors of PRMT5 and has been found to be pharmacologically active. Here, crystalline free bases of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile have been found that are suitable for use in pharmaceutical compositions.

### SUMMARY OF THE INVENTION

In one aspect, this disclosure provide crystalline forms of the free base of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile, shown below, and hereinafter Compound 1.

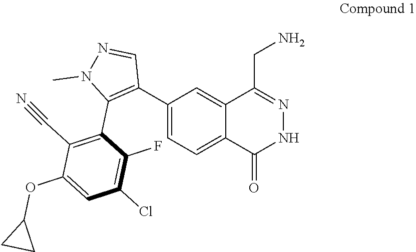

In another aspect, this disclosure also provides a particular crystalline form of Compound 1, hereinafter free base Form A. This disclosure further provides processes of preparing free base Form A. This disclosure further provides a pharmaceutical composition comprising free base Form A and a pharmaceutically acceptable carrier.

In another aspect, this disclosure also provides a particular crystalline form of Compound 1, hereinafter free base Form B. This disclosure further provides processes of preparing free base Form B. This disclosure further provides a pharmaceutical composition comprising free base Form B and a pharmaceutically acceptable carrier.

In another aspect, this disclosure also provides a particular crystalline form of Compound 1, hereinafter free base Form C. This disclosure further provides processes of preparing free base Form C. This disclosure further provides a pharmaceutical composition comprising free base Form C and a pharmaceutically acceptable carrier.

In another aspect, this disclosure also provides a particular crystalline form of Compound 1, hereinafter free base Form D. This disclosure further provides processes of preparing free base Form D.

This disclosure further provides a pharmaceutical composition comprising free base Form D and a pharmaceutically acceptable carrier.

This disclosure further provides methods of treating cancer comprising administering to a subject in need of such treatment a crystalline form of Compound 1 as disclosed herein or a pharmaceutical composition comprising a crystalline form of Compound 1.

### BRIEF DESCRIPTION OF THE FIGURES

FIG. 1A is a differential scanning calorimetric (DSC) thermogram and a thermal gravimetric analysis (TGA) overlay of free base Form A.

FIG. 1B is an X-ray powder diffraction (XRPD) pattern of free base Form A.

FIG. 2A is a differential scanning calorimetric (DSC) thermogram and a thermal gravimetric analysis (TGA) overlay of free base Form B.

FIG. 2B is an X-ray powder diffraction (XRPD) pattern of free base Form B.

FIG. 3A is a differential scanning calorimetric (DSC) thermogram and a thermal gravimetric analysis (TGA) overlay of free base Form C.

FIG. 3B is an X-ray powder diffraction (XRPD) pattern of free base Form C.

FIG. 4A is a differential scanning calorimetric (DSC) thermogram and a thermal gravimetric analysis (TGA) overlay of free base Form D.

FIG. 4B is an X-ray powder diffraction (XRPD) pattern of free base Form D.

FIG. 5A is a graph area under the curve for plasma concentrations for Free Base Form A administered in mammals.

FIG. 5B is a graph area under the curve for plasma concentrations for Free Base Form A administered in mammals.

FIG. 6 is a graph of the total impurity content vs. excipient for Free Base Form A of Compound 1.

FIG. 7A is a graph of the porosity vs. compaction pressure for Free Base Form A of Compound 1.

FIG. 7B is a graph of the tensile strength vs. compaction pressure for Free Base Form A of Compound 1.

FIG. 7C is a graph of the tensile strength vs. porosity for Free Base Form A of Compound 1.

FIG. 8 X-ray powder diffraction (XRPD) pattern of Free base Form A of Compound 1 before and after compression at high force.

FIG. 9A is a graph of the intrinsic dissolution rate of Free Base Form A of Compound 1.

FIG. 9B is a graph of the dissolution of Free Base Form A of Compound 1 in a capsule in biorelevant media.

### DETAILED DESCRIPTION OF THE INVENTION

As noted above, the invention provides particular crystalline forms of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile free base.

In some embodiments, the invention also provides free base Form A, i.e., a particular crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile free base.

In some embodiments, the invention also provides free base Form B, i.e., a particular crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile free base.

In some embodiments, the invention also provides free base Form C, i.e., a particular crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile free base.

In some embodiments, the invention also provides free base Form D, i.e., a particular crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile free base.

The crystalline forms as described herein may be characterized using a number of methods known to the person of ordinary skill in the art including thermal analysis (e.g., differential scanning calorimetry (DSC), thermal gravimetric analysis (TGA)), X-ray powder diffraction (XRPD), microscopy (e.g., scanning electron microscopy (SEM), polarized light microscopy), and spectroscopy (e.g., infrared, Raman, solid-state nuclear magnetic resonance, proton nuclear magnetic resonance (¹HNMR). The particle size and size distribution may be determined by conventional methods, such as laser light scattering technique. The purity of the crystalline forms provided herein may be determined by standard analytical methods, such as thin layer chromatography (TLC), gel electrophoresis, gas chromatography, high performance liquid chromatography (HPLC), and mass spectroscopy (MS).

### Free Base Form A

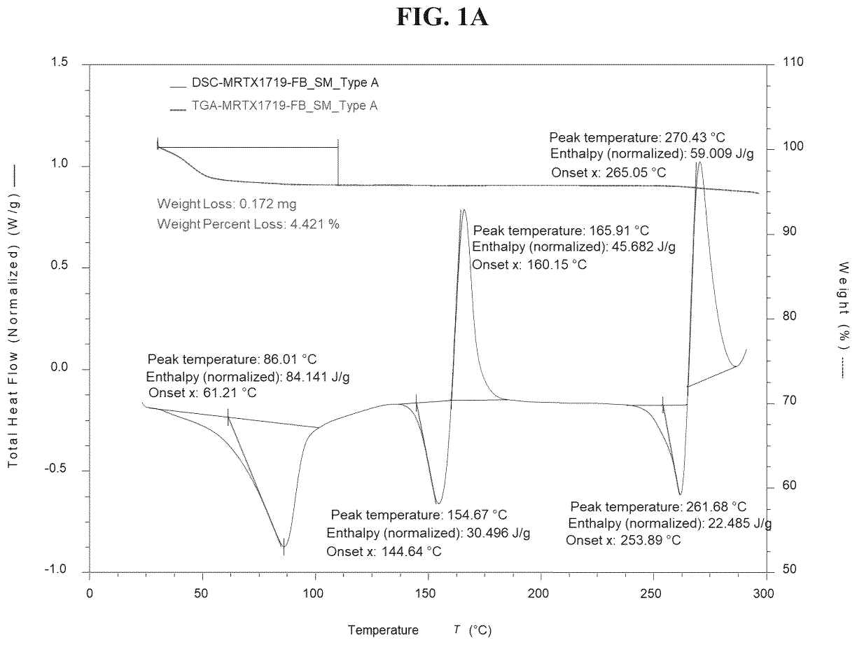

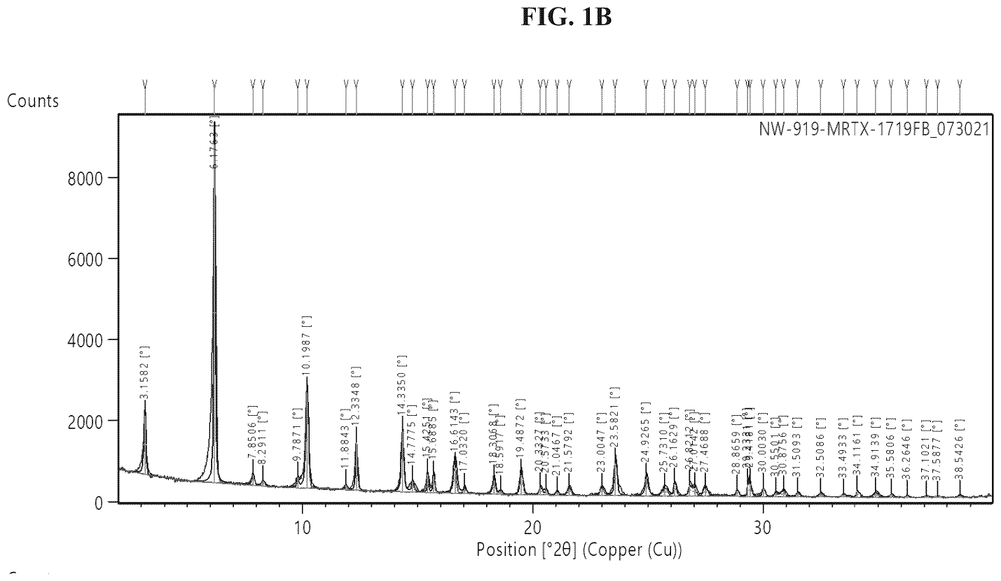

In one embodiment this disclosure provides a crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile free base, i.e., free base Form A. In various embodiments, free base Form A has a differential scanning calorimetric (DCS) thermogram. In some embodiments as described herein, free base Form A has a DSC thermogram with both exothermic and endothermic peaks. For example, in some embodiments, free base Form A has an endothermic DSC peak temperature of about 86° C., e.g., within about ±2% of 86° C. In some embodiments, free base Form A has an endothermic DSC peak temperature within ±1% of 86° C. or within ±0.5% of 86° C. In some embodiments, free base Form A has an endothermic DSC peak temperature of about 154° C., e.g., within about ±2% of 154° C. In some embodiments, free base Form A has an endothermic DSC peak temperature within ±1% of 154° C. or within ±0.5% of 154° C. In some embodiments, free base Form A has an exothermic DSC peak temperature of about 166° C., e.g., within about ±2% of 166° C. In some embodiments, free base Form A has an exothermic DSC peak temperature within ±1% of 166° C. or within ±0.5% of 166° C. In some embodiments, free base Form A has an endothermic DSC peak temperature of about 261° C., e.g., within about ±2% of 261° C. In some embodiments, free base Form A has an endothermic DSC peak temperature within ±1% of 261° C. or within ±0.5% of 261° C. In some embodiments, free base Form A has an exothermic DSC peak temperature of about 270° C., e.g., within about ±2% of 270° C. In some embodiments, free base Form A has an exothermic DSC peak temperature within ±1% of 270° C. or within ±0.5% of 270° C. For example, in some embodiments, free base Form A has a DSC thermogram substantially shown in FIG. 1A.

In various embodiments, free base Form A has an X-ray powder diffraction (XRPD) pattern. In some embodiments, free base Form A has an XRPD pattern comprising a peak at a two-theta angle of 3.1°±0.2°. In some embodiments, free base Form A has an XRPD pattern comprising a peak at a two-theta angle of 6.2°±0.2°. In some embodiments, free base Form A has an XRPD pattern comprising a peak at a two-theta angle of 10.2°±0.2°. In some embodiments, free base Form A has an XRPD pattern comprising a peak at a two-theta angle of 14.3°±0.2°. In some embodiments, free base Form A has an XRPD pattern comprising peaks at a two-theta angles of 3.1°±0.2°, 6.2°±0.2°, 10.2°±0.2°, and 14.3°±0.2°. For example, in some embodiments, free base Form A has an XRPD pattern substantially shown in FIG. 1B. In some embodiments as described herein, free base Form A is a sesquihydrate.

In some embodiments as described herein, free base Form A has a thermal gravimetric analysis (TGA) plot comprising a mass loss of about 4.4% when heated from about 25° C. to about 150° C. For example, in some embodiments, free base Form A has a TGA plot substantially shown in FIG. 1A.

In some embodiments as described herein, free base Form A has a purity of at least 97% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile free base. In some embodiments, free base Form A has a purity of at least 98% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile free base

### Free Base Form B

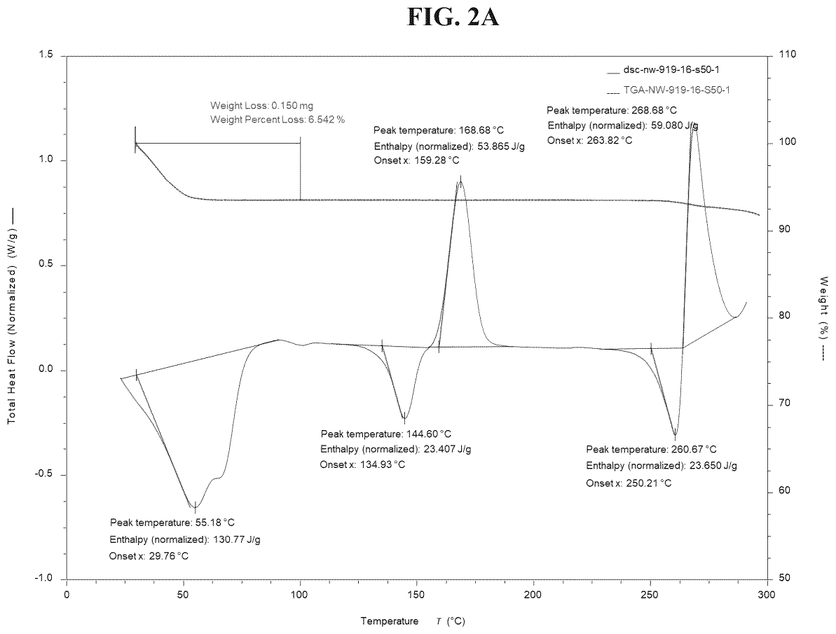

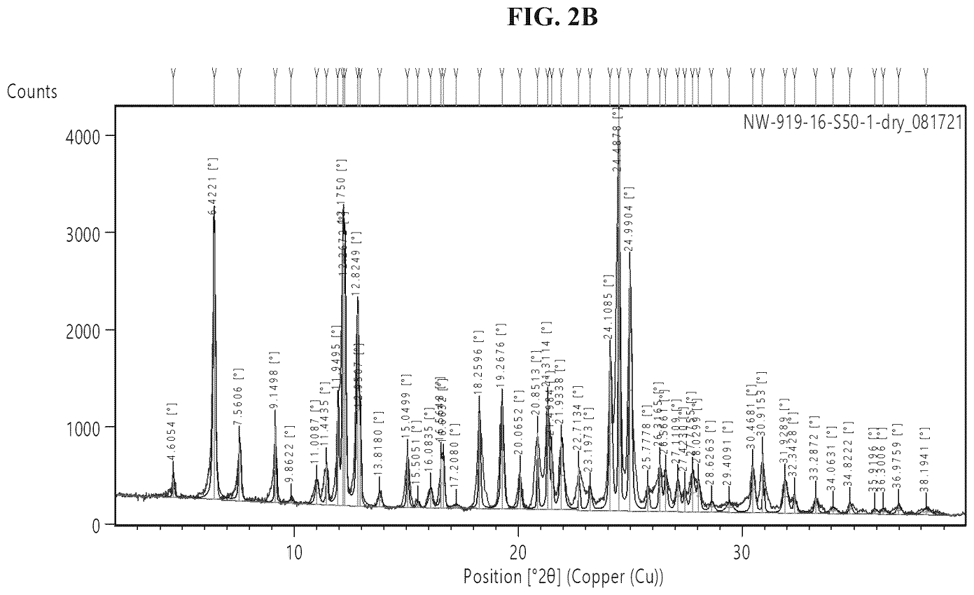

Another embodiment this disclosure as described herein provides a crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile free base, i.e., free base Form B. In various embodiments, free base Form B has a differential scanning calorimetric (DCS) thermogram. In some embodiments as described herein, free base Form B has a DSC thermogram with both exothermic and endothermic peaks. For example, in some embodiments, free base Form B has an endothermic DSC peak temperature of about 55° C., e.g., within about ±2% of 55° C. In some embodiments, free base Form B has an endothermic DSC peak temperature within ±1% of 55° C. or within ±0.5% of 55° C. In some embodiments, free base Form B has an endothermic DSC peak temperature of about 144° C., e.g., within about ±2% of 144° C. In some embodiments, free base Form B has an endothermic DSC peak temperature within ±1% of 144° C. or within ±0.5% of 144° C. In some embodiments, free base Form B has an exothermic DSC peak temperature of about 168° C., e.g., within about ±2% of 168° C. In some embodiments, free base Form B has an exothermic DSC peak temperature within ±1% of 168° C. or within ±0.5% of 168° C. In some embodiments, free base Form B has an endothermic DSC peak temperature of about 260° C., e.g., within about ±2% of 260° C. In some embodiments, free base Form B has an endothermic DSC peak temperature within ±1% of 260° C. or within ±0.5% of 260° C. In some embodiments, free base Form B has an exothermic DSC peak temperature of about 268° C., e.g., within about ±2% of 268° C. In some embodiments, free base Form B has an exothermic DSC peak temperature within ±1% of 268° C. or within ±0.5% of 268° C. For example, in some embodiments, free base Form B has a DSC thermogram substantially shown in FIG. 2A.

In various embodiments, free base Form B has an X-ray powder diffraction (XRPD) pattern. In some embodiments, the crystalline form free base Form B has an XRPD pattern comprising a peak at a two-theta angle of 6.4°±0.2°. In some embodiments, free base Form B has an XRPD pattern comprising a peak at a two-theta angle of 12.2°±0.2°. In some embodiments, free base Form B has an XRPD pattern comprising a peak at a two-theta angle of 12.8°±0.2°. In some embodiments, free base Form B has an XRPD pattern comprising a peak at a two-theta angle of 24.5°±0.2°. In some embodiments, free base Form B has an XRPD pattern comprising a peak at a two-theta angle of 25.0°±0.2°. In some embodiments, free base Form B has an XRPD pattern comprising peaks at a two-theta angles of 6.4°±0.2°, 12.2°±0.2°, 12.8°±0.2°, 24.5°±0.2°, and 25.0°±0.2°. For example, in some embodiments, free base Form B has an XRPD pattern substantially shown in FIG. 2B. In some embodiments as described herein, free base Form B is a dihydrate.

In some embodiments as described herein, free base Form B has a thermal gravimetric analysis (TGA) plot comprising a mass loss of about 6.5% when heated from about 25° C. to about 150° C. For example, free base Form B has a TGA plot substantially shown in FIG. 2A.

In some embodiments as described herein, free base Form B has a purity of at least 97% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile free base. In some embodiments, free base Form B has a purity of at least 98% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile free base.

### Free Base Form C

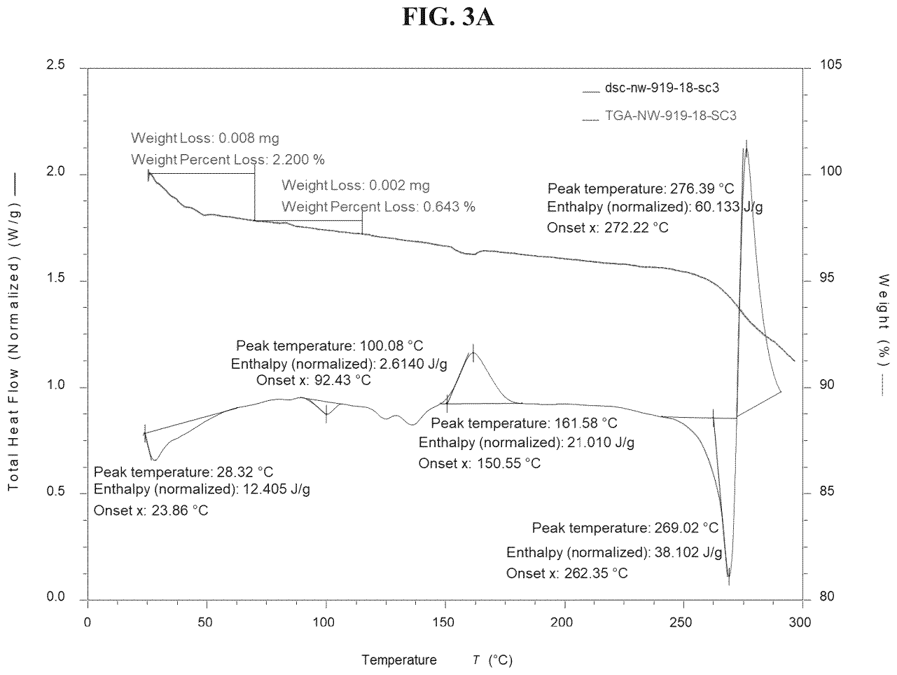

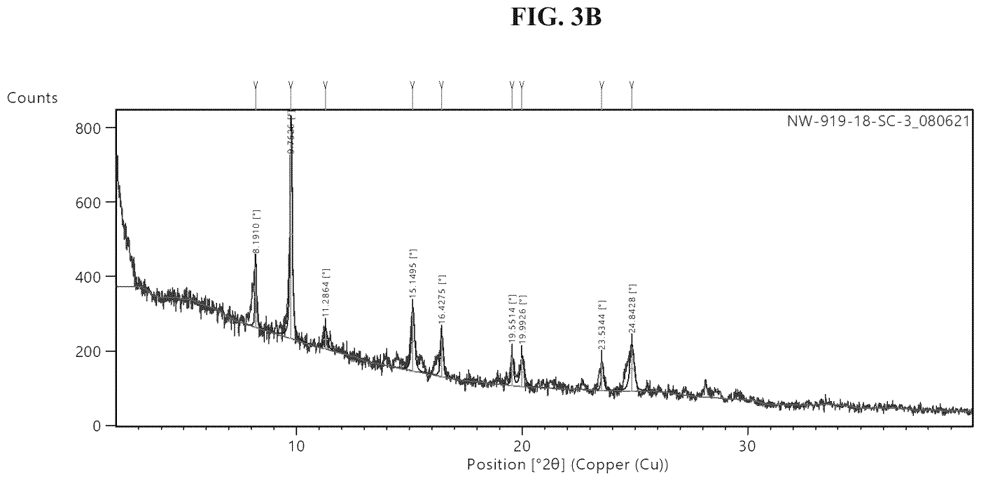

Another embodiment this disclosure as described herein provides a crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile free base, i.e., free base Form C. In various embodiments, free base Form C has a differential scanning calorimetric (DCS) thermogram. In some embodiments as described herein, free base Form C has a DSC thermogram with both exothermic and endothermic peaks. For example, in some embodiments, free base Form C has an endothermic DSC peak temperature of about 28° C., e.g., within about ±2% of 28° C. In some embodiments, free base Form C has an endothermic DSC peak temperature within ±1% of 28° C. or within ±0.5% of 28° C. In some embodiments, free base Form C has an endothermic DSC peak temperature of about 100° C., e.g., within about ±2% of 100° C. In some embodiments, free base Form C has an endothermic DSC peak temperature within ±1% of 100° C. or within ±0.5% of 100° C. In some embodiments, free base Form C has an exothermic DSC peak temperature of about 161° C., e.g., within about ±2% of 161° C. In some embodiments, free base Form C has an exothermic DSC peak temperature within ±1% of 161° C. or within ±0.5% of 161° C. In some embodiments, free base Form C has an endothermic DSC peak temperature of about 269° C., e.g., within about ±2% of 269° C. In some embodiments, free base Form C has an endothermic DSC peak temperature within ±1% of 269° C. or within ±0.5% of 269° C. In some embodiments, free base Form C has an exothermic DSC peak temperature of about 276° C., e.g., within about ±2% of 276° C. In some embodiments, free base Form C has an exothermic DSC peak temperature within ±1% of 276° C. or within ±0.5% of 276° C. For example, in some embodiments, free base Form C has a DSC thermogram substantially shown in FIG. 3A.

In various embodiments, free base Form C has an X-ray powder diffraction (XRPD) pattern. In some embodiments, the crystalline form free base Form C has an XRPD pattern comprising a peak at a two-theta angle of 8.2°±0.2°. In some embodiments, free base Form C has an XRPD pattern comprising a peak at a two-theta angle of 9.7°±0.2°. In some embodiments, free base Form C has an XRPD pattern comprising a peak at a two-theta angle of 15.1°±0.2°. In some embodiments, free base Form C has an XRPD pattern comprising a peak at a two-theta angle of 24.8°±0.2°. In some embodiments, free base Form C has an XRPD pattern comprising peaks at a two-theta angles of 8.2°±0.2°, 9.7°±0.2°, 15.1°±0.2°, and 24.8°±0.2°. For example, in some embodiments, free base Form C has an XRPD pattern substantially shown in FIG. 3B. In some embodiments as described herein, free base Form C is a channel hydrate, e.g., solvate, racemate.

In some embodiments as described herein, free base Form C has a thermal gravimetric analysis (TGA) plot comprising a mass loss of about 3.3% when heated from about 25° C. to about 200° C. For example, in some embodiments, free base Form C has a TGA plot substantially shown in FIG. 3A.

In some embodiments as described herein, free base Form C has a purity of at least 97% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile free base. In some embodiments, free base Form C has a purity of at least 98% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile free base.

### Free Base Form D

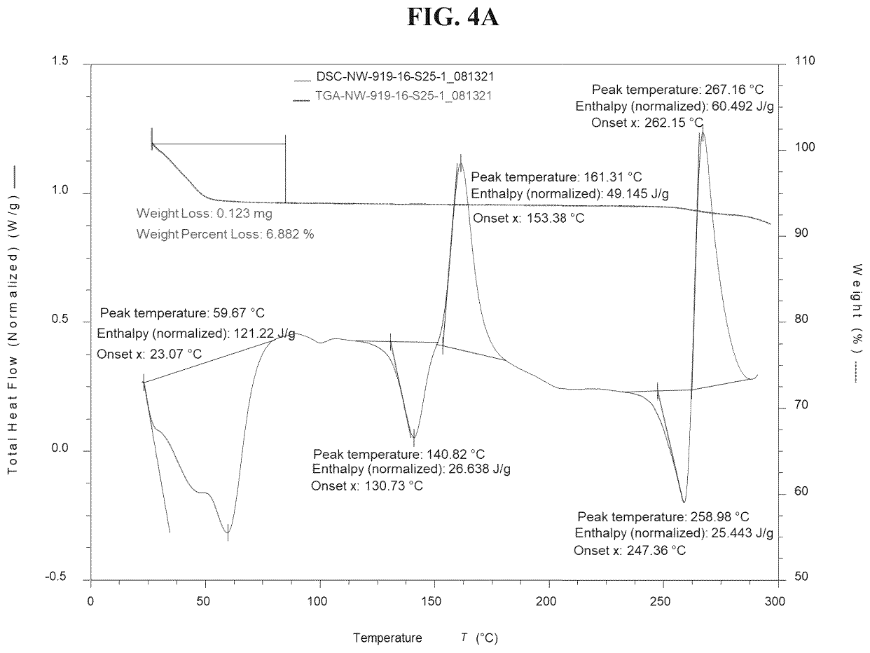

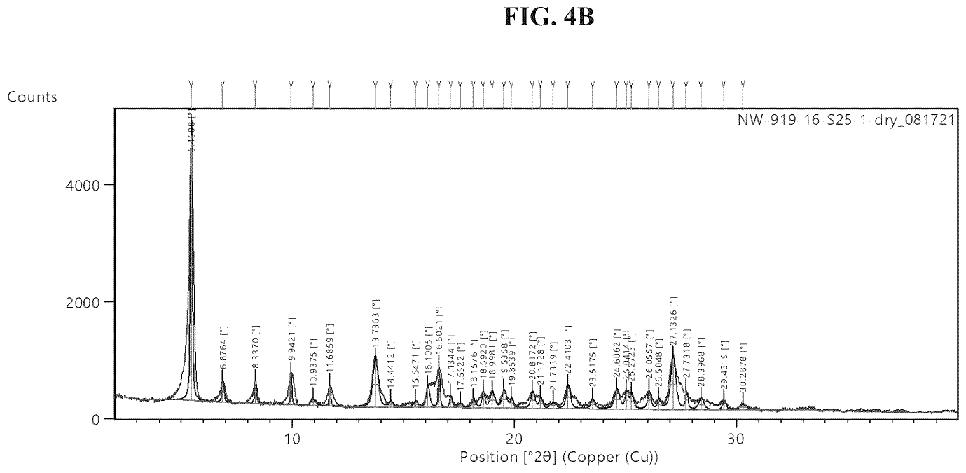

Another embodiment this disclosure as described herein provides a crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile free base, i.e., free base Form D. In various embodiments, free base Form D has a differential scanning calorimetric (DCS) thermogram. In some embodiments as described herein, free base Form D has a DSC thermogram with both exothermic and endothermic peaks. For example, in some embodiments, free base Form D has an endothermic DSC peak temperature of about 59° C., e.g., within about ±2% of 59° C. In some embodiments, free base Form D has an endothermic DSC peak temperature within ±1% of 59° C. or within ±0.5% of 59° C. In some embodiments, free base Form D has an endothermic DSC peak temperature of about 140° C., e.g., within about ±2% of 140° C. In some embodiments, free base Form D has an endothermic DSC peak temperature within ±1% of 140° C. or within ±0.5% of 140° C. In some embodiments, free base Form D has an exothermic DSC peak temperature of about 161° C., e.g., within about ±2% of 161° C. In some embodiments, free base Form D has an exothermic DSC peak temperature within ±1% of 161° C. or within ±0.5% of 161° C. In some embodiments, free base Form D has an endothermic DSC peak temperature of about 258° C., e.g., within about ±2% of 258° C. In some embodiments, free base Form D has an endothermic DSC peak temperature within ±1% of 258° C. or within ±0.5% of 258° C. In some embodiments, free base Form D has an exothermic DSC peak temperature of about 267° C., e.g., within about ±2% of 267° C. In some embodiments, free base Form D has an exothermic DSC peak temperature within ±1% of 267° C. or within ±0.5% of 267° C. For example, in some embodiments, free base Form D has a DSC thermogram substantially show in FIG. 4A.

In various embodiments, free base Form D has an X-ray powder diffraction (XRPD) pattern. In some embodiments, free base Form D has an XRPD pattern comprising a peak at a two-theta angle of 5.4°±0.2°. In some embodiments, free base Form D has an XRPD pattern comprising a peak at a two-theta angle of 9.9°±0.2°. In some embodiments, free base Form D has an XRPD pattern comprising a peak at a two-theta angle of 13.7°±0.2°. In some embodiments, free base Form D has an XRPD pattern comprising a peak at a two-theta angle of 27.1°±0.2°. In some embodiments, free base Form C has an XRPD pattern comprising peaks at a two-theta angles of 5.4°±0.2°, 9.9°±0.2°, 13.7°±0.2°, and 27.1°±0.2°. For example, in some embodiments, free base Form D has an XRPD pattern substantially shown in FIG. 4B. In some embodiments as described herein, free base Form D is a hydrate.

In some embodiments as described herein, free base Form D has a thermal gravimetric analysis (TGA) plot comprising a mass loss of about 6.9% when heated from about 25° C. to about 150° C. For example, in some embodiments, free base Form D has a TGA plot substantially shown in FIG. 4A.

In some embodiments as described herein, free base Form D has a purity of at least 97% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile free base. In some embodiments, free base Form D has a purity of at least 98% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile free base.

### Processes of Preparing Crystalline Forms of Free Base of Compound 1

In another aspect, this disclosure provides processes of preparing crystalline forms of free bases of Compound 1, e.g., free base Form A, free base Form B, free base Form C, and free base Form D. Crystalline forms of free base Form A, free base Form B, free base Form C, and free base Form D can be made by a variety of methods as discussed in the Examples below. For example, the crystalline forms as described herein may be prepared by slurry methods, anti-solvent addition, solid vapor diffusion, liquid vapor diffusion, slow evaporation at room temperature, slow cooling, or polymer induced crystallization.

In various embodiments, the slurry method can be conducted at a variety of temperatures and with a variety of solvents. For example, in some embodiments, the slurry method is conducted at room temperature or at an elevated temperature (e.g., 50° C.). To prepare crystalline forms with the slurry method, free base of Compound 1 is suspended and stirred in a solvent at a temperature (e.g., room temperature or elevated temperature) to provide solids.

In various embodiments, the anti-solvent addition method can be conducted at a variety of temperatures and with a variety of solvents. For example, in some embodiments, the anti-solvent addition method is conducted at room temperature. To prepare crystalline forms with the anti-solvent addition method, free base of Compound 1 is dissolved in a solvent to obtain a saturated solution and an anti-solvent is added in an amount of up to, for example, 20 times in volume to provide solids.

In various embodiments, the solid vapor diffusion method can be conducted at a variety of temperatures and with a variety of solvents. For example, in some embodiments, the solid vapor diffusion method is conducted at room temperature. To prepare crystalline forms with the solid vapor diffusion method, free base of Compound 1 is provided in a first vial which is placed in a second vial containing a solvent. There is no physical contact between solid free base of Compound 1 in the first vial and the solvent in the second vial. The solids are then characterized after 14 days.

In various embodiments, the liquid vapor diffusion method can be conducted at a variety of temperatures and with a variety of solvents. For example, in some embodiments, the liquid vapor diffusion method is conducted at room temperature. To prepare crystalline forms with the liquid vapor diffusion method, free base of Compound 1 is dissolved in a solvent to obtain a saturated solution in a first vial and the first vial is then placed in a second vial containing an anti-solvent to provide solids.

In various embodiments, the slow evaporation method can be conducted at a variety of temperatures and with a variety of solvents. For example, in some embodiments, the slow evaporation method is conducted at room temperature or at elevated temperatures (e.g., 50° C.). To prepare crystalline forms with the slow evaporation method, free base of Compound 1 is dissolved in a solvent to obtain a saturated solution. The vial is then covered with paraffin film with a plurality, e.g., 3-5, holes and allowed to evaporate to provide solids.

In various embodiments, the slow cooling method can be conducted at a variety of temperatures and with a variety of solvents. For example, in some embodiments, the slow cooling method is conducted at elevated temperatures (e.g., 55° C.). To prepare crystalline forms with the slow cooling method, free base of Compound 1 is dissolved in a solvent to obtain a saturated solution at, for example, in the range of 40-70° C., or 45-70° C., or 50-70° C., or 40-65° C., or 45-65° C., or 50-65° C., or 40-60° C., or 45-60° C., or 50-60° C. In some embodiments, to prepare the crystalline forms with the slow cooling method, free base of Compound 1 is dissolved in a solvent to obtain a saturated solution, for example, at approximately 55° C. The solution is slowly cooled down to room temperature to provide solids.

In various embodiments, the polymer induced crystallization method can be conducted at a variety of temperatures and with a variety of solvents. For example, in some embodiments, the polymer induced crystallization is conducted at room temperature. To prepare crystalline forms with the polymer induced crystallization method, free base of Compound 1 is dissolved in a solvent to provide a saturated solution. A polymer is then added to the saturated solution to induce heteronucleation and provide solids.

In some embodiments, to provide free base Form A, slurry methods, anti-solvent addition, solid vapor diffusion, liquid vapor diffusion, or slow cooling methods may be used. When a slurry method is used, the solvent may be selected from isopropyl alcohol (IPA), ethyl acetate (EtOAc), isopropyl acetate (IPAc), chloroform (CHCl₃), dimethoxyethane, methyl tert-butyl ether (MTBE), acetonitrile (ACN), anisole, cyclopentyl methyl ether (CPME), toluene, tetrahydrofuran (THF)/water, 2-methyl tetrahydrofuran (2-MeTHF), or n-heptane. When an anti-solvent addition method is used, the solvent may be selected from THF, dimethyl sulfoxide (DMSO), and dichloromethane (DCM) and the anti-solvent may be selected from water and heptane. When solid vapor diffusion is used, the solvent may be selected from ethanol, 1-butanol, 2Me-THF, dimethoxyethane, MTBE, toluene, EtOAc, IPAc, and H₂O. When liquid vapor diffusion is used, the solvent may be n-methylpyrrolidone (NMP) and the anti-solvent may be water. When slow cooling method is used, the solvent may be ACN.

In some embodiments, to provide free base Form B, slurry methods, slow cooling, or polymer induced crystallization methods may be used. When a slurry method is used, the solvent may be selected from ethanol (EtOH), IPA, methyl isobutyl ketone (MIBK), and methanol (MeOH). When slow cooling method is used, the solvent may be EtOH. When polymer induced crystallized is used, the solvent may be 2-butanol and the polymer may be hydroxypropyl methylcellulose (HPMC).

In some embodiments, to form free base Form C, solid vapor diffusion, liquid vapor diffusion, slow evaporation, or slow cooling methods may be used. When solid vapor diffusion is used, the solvent may be selected from ACN and 1,4-dioxane. When liquid vapor diffusion is used, the solvent may be NMP and the anti-solvent may be IPA or MTBE. When slow evaporation is used, the solvent may be ACN, IPAc, 2-MeTHF. When slow cooling method is used, the solvent may be 2-MeTHF, MeOH, and IPAc.

In some embodiments, to provide free base Form D, slurry methods or polymer induced crystallization methods may be used. When a slurry method is used, the solvent may be selected from MeOH. When polymer induced crystallized is used, the solvent may be MeOH and the polymer may be polyvinylpyrrolidone (PVP) or polyvinyl alcohol (PVA).

### Pharmaceutical Compositions

In another aspect, this disclosure provides pharmaceutical comprisings a crystalline form of Compound 1 (e.g., free base Form A, free base Form B, free base Form C, and free base Form D) and an appropriate carrier, excipient or diluent. The exact nature of the carrier, excipient or diluent will depend upon the desired use for the composition, and may range from being suitable or acceptable for veterinary uses to being suitable or acceptable for human use. The composition may optionally include one or more additional compounds. In certain embodiments, the composition may include one or more antibiotic compounds. In another aspect, this disclosure provides pharmaceutical compositions comprising free base Form A and a pharmaceutically acceptable carrier. In another aspect, this disclosure provides pharmaceutical compositions comprising free base Form B and a pharmaceutically acceptable carrier. In another aspect, this disclosure provides pharmaceutical compositions comprising free base Form C and a pharmaceutically acceptable carrier. In another aspect, this disclosure provides pharmaceutical compositions comprising free base Form D and a pharmaceutically acceptable carrier.

When used to treat or prevent such diseases, Compound 1 described herein may be administered singly, as mixtures of one or more compounds or in mixture or combination with other agents useful for treating such diseases and/or the symptoms associated with such diseases. The compounds may also be administered in mixture or in combination with agents useful to treat other disorders or maladies, such as steroids, membrane stabilizers, 5LO inhibitors, leukotriene synthesis and receptor inhibitors, inhibitors of IgE isotype switching or IgE synthesis, IgG isotype switching or IgG synthesis, β-agonists, tryptase inhibitors, aspirin, COX inhibitors, methotrexate, anti-TNF drugs, retuxin, PD4 inhibitors, p38 inhibitors, PDE4 inhibitors, and antihistamines, to name a few. The Compound 1 may be administered in the crystalline forms as described herein, or as pharmaceutical compositions comprising the crystalline forms as described herein.

Pharmaceutical compositions comprising the various crystalline forms of Compound 1 may be manufactured by means of conventional mixing, dissolving, granulating, dragee-making levigating, emulsifying, encapsulating, entrapping or lyophilization processes. The compositions may be formulated in conventional manner using one or more physiologically acceptable carriers, diluents, excipients or auxiliaries which facilitate processing of the compounds into preparations which can be used pharmaceutically.

Pharmaceutical compositions may take a form suitable for virtually any mode of administration, including, for example, topical, ocular, oral, buccal, systemic, nasal, injection, transdermal, rectal, vaginal, etc., or a form suitable for administration by inhalation or insufflation.

For topical administration, the compound(s) may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives. Alternatively, the injectable formulation may be provided in powder form for reconstitution with a suitable vehicle, including but not limited to sterile pyrogen free water, buffer, dextrose solution, etc., before use. To this end, the active compound(s) may be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art with, for example, sugars, films or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, Cremophore™ or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Preparations for oral administration may be suitably formulated to give controlled release of the compound, as is well known. For buccal administration, the compositions may take the form of tablets or lozenges formulated in conventional manner. For rectal and vaginal routes of administration, the compound(s) may be formulated as solutions (for retention enemas) suppositories or ointments containing conventional suppository bases such as cocoa butter or other glycerides.

For nasal administration or administration by inhalation or insufflation, the compound(s) can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch.

For ocular administration, the compound(s) may be formulated as a solution, emulsion, suspension, etc. suitable for administration to the eye. A variety of vehicles suitable for administering compounds to the eye are known in the art.

For prolonged delivery, the compound(s) can be formulated as a depot preparation for administration by implantation or intramuscular injection. The compound(s) may be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the compound(s) for percutaneous absorption may be used. To this end, permeation enhancers may be used to facilitate transdermal penetration of the compound(s).

Alternatively, other pharmaceutical delivery systems may be employed. Liposomes and emulsions are well-known examples of delivery vehicles that may be used to deliver compound(s). Certain organic solvents such as dimethyl sulfoxide (DMSO) may also be employed, although usually at the cost of greater toxicity.

The pharmaceutical compositions may, if desired, be presented in a pack or dispenser device which may contain one or more unit dosage forms containing the compound(s). The pack may, for example, comprise metal or plastic foil, such as a blister pack. The pack or dispenser device may be accompanied by instructions for administration.

### Methods of Use

The crystalline forms described herein, or compositions thereof, will generally be used in an amount effective to achieve the intended result, for example in an amount effective to treat or prevent the particular disease being treated. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also generally includes halting or slowing the progression of the disease, regardless of whether improvement is realized.

In yet another aspect, the invention provides for methods for inhibiting PRMT5 activity in a cell, comprising contacting the cell in which inhibition of PRMT5 activity is desired in vitro with an effective amount of a crystalline forms of Compound 1 as described herein or pharmaceutical compositions containing the crystalline forms of Compound 1 as described herein. In one embodiment, the cell is an MTAP-deficient cell.

The compositions and methods provided herein are particularly deemed useful for inhibiting PRMT5 activity in a cell in vivo. In one embodiment, a cell in which inhibition of PRMT5 activity is desired is contacted in vivo with a therapeutically effective amount of crystalline forms of Compound 1 as described herein or pharmaceutical compositions containing the crystalline forms of Compound 1 as described herein. In one embodiment, the cell is an MTAP-deficient cell. In one embodiment, the negatively modulating the activity of PRMT5 occurs in the presence of bound MTA.

By negatively modulating the activity of PRMT5, particularly in cases for cells that lack MTAP activity, the methods are designed to inhibit PRMT5 activity to block cellular proliferation. The cells may be contacted in a single dose or multiple doses in accordance with a particular treatment regimen to affect the desired negative modulation of PRMT5. The degree PRMT5 inhibition may be monitored in vitro against the enzyme in the presence and absence of MTA and in the cell using well known methods, including those described in Example B below, to assess the effectiveness of treatment and dosages.

In another aspect, methods of treating cancer comprising administering to a patient having cancer a therapeutically effective amount of crystalline forms of Compound 1 as described herein or pharmaceutical compositions containing the crystalline forms of Compound 1 as described herein. In one embodiment, the cancer is an MTAP-associated cancer.

The compositions and methods provided herein may be used for the treatment of a wide variety of cancer including tumors such as prostate, breast, brain, skin, cervical carcinomas, testicular carcinomas, etc. More particularly, cancers that may be treated by the compositions and methods of the invention include, but are not limited to tumor types such as astrocytic, breast, cervical, colorectal, endometrial, esophageal, gastric, head and neck, hepatocellular, laryngeal, lung, oral, ovarian, prostate and thyroid carcinomas and sarcomas. More specifically, these compounds can be used to treat: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma. In certain embodiments, the cancer is diffuse large B-cell lymphoma (DLBCL).

In one embodiment, the cancer is an MTAP-associated cancer selected from hepatocellular carcinoma, breast cancer, skin cancer, bladder cancer, liver cancer, pancreatic cancer, and head and neck cancer.

In other embodiments, the cancer is selected from the group consisting of ovarian serous cystadenocarcinoma, squamous cell lung cancer, lung adenocarcinoma, mesothelioma; esophogeal squamous cell carcinoma, gastric adenocarcinoma, pancreatic ductal adenocarcinoma, kidney adenocarcinoma, bladder transitional cell carcinoma, hepatocellular carcinoma, cholangiocarcinoma, cholangiocarcinoma; osteosarcoma, multiple myeloma, astrocytoma, glioma, glioblastoma, uterine sarcoma, acute myeloid leukemia, acute lymphoblastic leukemia, non-Hodgkin's lymphoma, malignant melanoma, endometrial carcinoma and thyroid carcinoma.

In other embodiments, the cancer is selected from the group consisting of bladder cancer, bone cancer, brain cancer, blood cancer, breast cancer, colorectal cancer, esophageal cancer, gastric cancer, head and neck cancer, kidney cancer, liver cancer, lung cancer, mesothelioma, ovarian cancer, pancreatic cancer, skin cancer, thyroid cancer and uterine cancer.

The concentration and route of administration to the patient will vary depending on the cancer to be treated. The crystalline forms of Compound 1 as described herein or pharmaceutical compositions containing the crystalline forms of Compound 1 as described herein also may be co-administered with other anti-neoplastic compounds, e.g., chemotherapy, or used in combination with other treatments, such as radiation or surgical intervention, either as an adjuvant prior to surgery or post-operatively.

### EXAMPLES

The following Examples are intended to illustrate further certain embodiments of the invention and are not intended to limit the scope of the invention.

### Example 1: Formation of Free Base Form a Crystalline Form of Compound 1

Compound 1 can be prepared as a gum according to procedures disclosed in published International Application No. WO2021050915. See Example 16-8.

Crystallization of Compound 1 was accomplished by slurring amorphous and/or mixtures of forms in isopropanol:water 9:1 v/v for 24 hours and isolating the resulting crystalline solid by filtration. The crystals produced were a white solid powder. The results of the characterization are summarized in Table 1 and shown in FIGS. 1A and 1B.

TABLE 1

| Sample | Free Base Form A |
|---|---|
| Crystallinity by XRPD | High |
| Wt. Loss in TGA (%, ° C.) | 4.4 (110) |
| Endotherm in DSC (peak, ° C.) | 86.01 |
| | 154.67 |
| | 261.68 |
| Exotherm. in DSC (peak, ° C.) | 165.91 |
| | 270.43 |
| Form | Sesquihydrate |

Table 2 describes the XRD pattern of Free Base Form A of Compound 1 shown in FIG. 1B.

TABLE 2

| Pos. (°2θ) | Intensity | Height (cts) |
|---|---|---|
| 3.1582 | Medium | 1613.32 |
| 6.1763 | High | 8529.13 |
| 7.8506 | Low | 268.12 |
| 9.7871 | Medium | 269.15 |
| 10.1987 | Low | 2423.48 |
| 12.3348 | Medium | 1209.26 |
| 14.3350 | Medium | 1530.38 |
| 14.7775 | Low | 288.38 |
| 15.4251 | Low | 467.43 |
| 15.6885 | Low | 418.69 |
| 16.6143 | Low | 648.9 |
| 18.3068 | Low | 355.94 |
| 19.4872 | Low | 506.85 |
| 23.5821 | Low | 812.97 |
| 24.9265 | Low | 436.53 |
| 26.1629 | Low | 318.15 |
| 26.8212 | Low | 264.88 |
| 29.2331 | Low | 304.84 |
| 29.4181 | Low | 328.8 |

### Example 2: Formation of Free Base Form B Crystalline Form of Compound 1

Approximately 30 mg of free base Form A, from Example 1, was suspended in 0.2 mL ethanol in a 4 mL vial to obtain a thick slurry. The slurry was stirred at 300-400 rpm at 50° C. for 6 days. A drop of the thick slurry was collected for analysis. The results of the characterization are summarized in Table 3 and shown in FIGS. 2A and 2B.

TABLE 3

| | |
|---|---|
| Sample | Free Base Form B |
| Crystallinity by XRPD | High |
| Wt. Loss in TGA (%, ° C.) | 6.5 (100) |
| Endotherm in DSC (peak, ° C.) | 55.18 |
| | 144.60 |
| | 260.67 |
| Exotherm in DSC (peak, ° C.) | 168.68 |
| | 268.68 |
| Form | Dihydrate |

Table 4 describes the XRD pattern of Free Base Form B of Compound 1 shown in FIG. 2B.

TABLE 4

| Pos. (°2θ) | Intensity | Height (cts) |
|---|---|---|
| 4.6054 | Low | 208.95 |
| 6.4221 | High | 2802.68 |
| 7.5606 | Low | 604.97 |
| 9.1498 | Medium | 798.81 |
| 11.0087 | Low | 230.29 |
| 11.4435 | Low | 425.25 |
| 11.9495 | Low | 1181.87 |
| 12.1750 | High | 2774.26 |
| 12.2672 | Low | 2221.19 |
| 12.8249 | High | 2019.66 |
| 12.9507 | Low | 852.19 |
| 15.0499 | Low | 543.55 |
| 16.0835 | Low | 199.31 |
| 16.5648 | Low | 523.97 |
| 16.6632 | Low | 499.37 |
| 18.2596 | Medium | 1013.08 |
| 19.2676 | Medium | 1095.25 |
| 20.0652 | Low | 392.02 |
| 20.8513 | Medium | 808.65 |
| 21.3114 | Medium | 1111.6 |
| 21.4984 | Medium | 597.66 |
| 21.9338 | Medium | 721.9 |
| 22.7134 | Low | 446.53 |
| 23.1973 | Low | 220.32 |
| 24.1085 | Medium | 1616.39 |
| 24.4878 | High | 4212.89 |
| 24.9904 | High | 2543.9 |
| 25.7778 | Low | 256.39 |
| 26.3165 | Low | 494.22 |
| 26.5661 | Low | 416.91 |
| 27.1109 | Low | 320.79 |
| 27.4230 | Low | 238.34 |
| 27.7755 | Low | 397.69 |
| 28.0299 | Low | 326.16 |
| 30.4681 | Low | 489.98 |
| 30.9153 | Low | 619.7 |
| 31.9289 | Low | 339.44 |

### Example 3: Formation of Free Base Form C Crystalline Form of Compound 1

Approximately 30 mg of free base Form A, from Example 1, was dissolved in 1.3 mL methanol at 55° C. in a 4 mL vial to obtain a clear solution. The solution was cooled down to 10° C. in the follow ramp: 55° C. for 3 hours, 40° C. for 4 hours, 25° C. for 4 hours, 15° C. for 5 hours, and 10° C. for 5 hours. White solids were crystallized out after the cooling cycle and collected for analysis. The results of the characterization are summarized in Table 5 and shown in FIGS. 3A and 3B.

TABLE 5

| | |
|---|---|
| Sample | Free Base Form C |
| Crystallinity by XRPD | Moderate |
| Wt. Loss in TGA (%, ° C.) | 3.3 (150) |
| Endotherm in DSC (peak, ° C.) | 28.32 |
| | 100.08 |
| | 269.02 |
| Exotherm in DSC (peak, ° C.) | 161.58 |
| | 276.39 |
| Form | Channel Hydrate/Solvate (Racemate) |

Table 6 describes the XRD pattern of Free Base Form C of Compound 1 shown

TABLE 6

| Pos. (°2θ) | Intensity | Height (cts) |
|---|---|---|
| 8.1910 | Medium | 165.34 |
| 9.7526 | High | 572.43 |
| 11.2864 | Low | 51.48 |
| 15.1495 | Medium | 163.22 |
| 16.4275 | Low | 106.38 |
| 19.5514 | Low | 76.61 |
| 19.9926 | Low | 75.95 |
| 23.5344 | Low | 79.52 |
| 24.8428 | Low | 130.5 |

### Example 4: Formation of Free Base Form D Crystalline Form of Compound 1

Approximately 30 mg of free base Form A, from Example 1, was suspended in 0.2 mL of methanol in a 4 mL vial to obtain a thick slurry. The slurry was stirred at 300-400 rpm at room temperature for 14 days. A drop of the thick slurry was collected for analysis. The results of the characterization are summarized in Table 7 and shown in FIGS. 4A and 4B.

TABLE 7

| | |
|---|---|
| Sample | Free Base Form D |
| Crystallinity by XRPD | High |
| Wt. Loss in TGA (%, ° C.) | 6.9 (85) |
| Endotherm in DSC (peak, ° C.) | 59.67 |
| | 140.82 |
| | 258.98 |
| Exotherm in DSC (peak, ° C.) | 161.30 |
| | 267.16 |
| Form | Hydrate |

Table 8 describes the XRD pattern of Free Base Form D of Compound 1 shown in FIG. 4B.

TABLE 8

| Pos. (°2θ) | Intensity | Height (cts) |
|---|---|---|
| 5.4500 | High | 4648.17 |
| 6.8764 | Low | 356.14 |
| 8.3370 | Low | 389.87 |
| 9.9421 | Low | 533.77 |
| 11.6859 | Medium | 355.66 |
| 13.7363 | Low | 805.73 |
| 16.1005 | Low | 346.39 |
| 16.6021 | Low | 690.75 |
| 18.5920 | Low | 276.92 |
| 18.9981 | Low | 263.32 |
| 19.5358 | Low | 292.26 |
| 20.8172 | Low | 269.61 |
| 21.1728 | Low | 179.28 |
| 22.4103 | Low | 384.45 |
| 24.6062 | Low | 325.83 |
| 25.0414 | Low | 301.52 |
| 25.2733 | Low | 257.66 |
| 26.0557 | Low | 309.47 |
| 27.1326 | Medium | 897.59 |
| 27.7318 | Low | 327.17 |

### Example 5: Relative Stability Investigation for Hydrate

The relative stability between free base Form A, Form B, and Form D was investigated by conducting competitive slurry experiments in isopropyl alcohol (IPA)/water systems at room temperature. As shown in Table 9, all forms were converted to Form B in pure IPA (α_{w}=0) after 1 day of stirring at room temperature. Further addition of Form A to the experiments verified the result. In systems with α_{w}≥0.37, only Form A was observed after 1 day of stirring at room temperature. Further addition of Form B and Form D to the experiment confirmed the outcome. At α_{w}=0.13, Form B and Form D were also converted to Form A eventually, while the conversion rate was slightly slower than those at higher water activities.

TABLE 9

| Trial | | | Crystal Form Resulting |
|---|---|---|---|
| No. | Solvent | Duration | from Stability Test |
| 1 | IPA | 1 d | Form B |
| | (α_{w} = 0.0) | 3 d (1 day after further | Form B |
| | | addition of Form A) | |
| | | 6 d (4 days after further | Form B |
| | | addition of Form A) | |
| 2 | IPA/H₂O | 1 d | Form B + Form A |
| | (α_{w} = 0.13) | | (mainly Form A) |
| | | 3 d (1 day after further | Form B + Form A |
| | | addition of Form B & D) | (mainly Form A) |
| | | 6 d (4 days after further | Form A |
| | | addition of Form B & D) | |
| 3 | IPA/H₂O | 1 d | Form A |
| | (α_{w} = 0.37) | 3 d (1 day after further | Form A |
| | | addition of Form B & D) | |
| | | 6 d (4 days after further | Form A |
| | | addition of Form B & D) | |
| 4 | IPA/H₂O | 1 d | Form A |
| | (α_{w} = 0.63) | 3 d (1 day after further | Form A |
| | | addition of Form B & D) | |
| | | 6 d (4 days after further | Form A |
| | | addition of Form B & D) | |
| 5 | IPA/H₂O | 1 d | Form A |
| | (α_{w} = 0.80) | 3 d (1 day after further | Form A |
| | | addition of Form B & D) | |
| | | 6 d (4 days after further | Form A |
| | | addition of Form B & D) | |
| 6 | H₂O | 1 d | Form A |
| | (α_{w} = 1.0) | 3 d (1 day after further | Form A |
| | | addition of Form B & D) | |
| | | 6 d (4 days after further | Form A |
| | | addition of Form B & D) | |

### Example 6: Other Processes to Prepare Free Bases Form a, Form B, Form C, and Form D of Compound 1

Other processes of preparing crystalline forms of Compound 1 were evaluated to provide free base Form A, free base Form B, free base Form C, and free base Form D. Eight different methods were used: slurry at room temperature, slurry at 50° C., anti-solvent addition, solid vapor diffusion, liquid vapor diffusion, slow evaporation at room temperature, slow cooling, and polymer induced crystallization.

For the slurry experiments at room temperature, approximately 30 mg of free base Form A of Compound 1, as discussed in Example 1, was suspended and stirred in different solvents (approximately 0.2 mL) in 4 mL vials at room temperature. The solids in the slurry were characterized by XRPD after 14 days. Table 10 discloses the parameters and resulting crystal forms for slurry experiments at room temperature.

TABLE 10

| Exp. | | | Crystal Form |
|---|---|---|---|
| No. | Solvent (v:v) | Observation | Obtained |
| 1 | MeOH | Thick slurry | Form D |
| 2 | IPA | Thick slurry | Form A |
| 3 | 1,4-Dioxane | Thin slurry | Form C (very small |
| | | | amount) |
| 4 | MIBK | Gel | N/A |
| 5 | MEK | Clear | N/A |
| 6 | EtOAc | Thick slurry | Form A |
| 7 | IPAc | Thick slurry | Form A |
| 8 | CHCl₃ | Medium slurry | Form A |
| 9 | Acetic acid | Clear | N/A |
| 10 | Dimethoxyethane | Medium slurry | Form A |
| 11 | MTBE | Thick slurry | Form A |
| 12 | ACN | Thick slurry | Form A |
| 13 | Anisole | Thick slurry | Form A |
| 14 | CPME | Thick slurry | Form A |
| 15 | Toluene | Thick slurry | Form A |
| 16 | THF | Thin slurry | Form C (very small |
| | | | amount) |
| 17 | THF/H₂O (α_{w} = 0.2) | Thick slurry | FB Form A |
| 18 | THF/H₂O (α_{w} = 0.4) | Thin slurry | FB Form A |
| 19 | THF/H₂O (α_{w} = 0.6) | Thick slurry | FB Form A |
| 20 | THF/H₂O (α_{w} = 0.8) | Thick slurry | FB Form A |
| 21 | H₂O | Thick slurry | FB Form A |

For the slurry experiments at 50° C., approximately 30 mg of free base Form A of Compound 1, as discussed in Example 1, was suspended and stirred in different solvents (approximately 0.2 mL) in 4 mL vials at 50° C. The solids in the slurry were characterized by XRPD after 6 days. Table 11 discloses the parameters and resulting crystal forms for slurry experiments at 50° C.

TABLE 11

| Exp. | | | Crystal Form |
|---|---|---|---|
| No. | Solvent (v:v) | Observation | Obtained |
| 22 | EtOH | White solids | Form B |
| 23 | IPA | White solids | Form B |
| 24 | 1,4-Dioxane | Thin slurry | Form C (very small |
| | | | amount) |
| 25 | Water | Thick slurry | Form A |
| 26 | MIBK | Thin slurry | Form B |
| 27 | MEK | Gel | Amorphous |
| 28 | EtOAc | White solids | Form A |
| 29 | IPAc | White solids | Form A |
| 30 | 2-MeTHF | White solids | Form A |
| 31 | Toluene | White solids | Form A |
| 32 | MTBE | White solids | Form A |
| 33 | ACN | White solids | Form A |
| 34 | THF/n-Heptane (1:1) | White solids | Form A |
| 35 | Acetone/Toluene (1:1) | Clear | N/A |
| 36 | MeOH | White solids | Form B |
| 37 | THF/H₂O (1:1) | Thick slurry | Form A |

For the anti-solvent addition experiments, approximately 30 mg of free base Form A of Compound 1, as discussed in Example 1, was dissolved in different solvents to obtain saturated solutions and anti-solvents were added in amounts of up to 20 times in volume. The obtained solids were characterized by XRPD. Table 12 discloses the parameters and resulting crystal forms of anti-solvent addition experiments.

TABLE 12

| Exp. ID | Solvent | Anti-solvent | Observation | Crystal Form Obtained |
|---|---|---|---|---|
| 38 | THF | H₂O | White solids | Form A |
| 39 | | MTBE | Clear | N/A |
| 40 | | Heptane | Gel | Amorphous |
| 41 | | IPA | Clear | N/A |
| 42 | DMSO | H₂O | White solids | Form A |
| 43 | | MTBE | Clear | N/A |
| 44 | | IPA | Clear | N/A |
| 45 | DCM | Heptane | White solids | Form A |
| 46 | | IPA | Clear | N/A |
| 47 | | CPME | Clear | N/A |
| 48 | Acetone | MTBE | Clear | N/A |
| 49 | | IPA | Clear | N/A |
| 50 | | water | Gel | Amorphous |
| 51 | | Heptane | Gel | Amorphous |

For the solid vapor diffusion experiments, approximately 30 mg of free base Form A of Compound 1, as discussed in Example 1, was kept in 4 mL vials which were placed in 20 mL glass vials containing different solvents. The solids were characterized by XRPD after 14 days. Table 13 discloses the parameters and resulting crystal forms of solid vapor diffusion experiments.

TABLE 13

| Exp. ID | Solvent | Observation | Crystal Form Obtained |
|---|---|---|---|
| 52 | EtOH | White solid | Form A |
| 53 | 1-Butanol | White solid | Form A |
| 54 | Acetone | Clear | N/A |
| 55 | MIBK | Gel | Poorly crystalline |
| 56 | 2Me-THF | White solids | Form A |
| 57 | Dimethoxyethane | White solids | Form A |
| 58 | MTBE | White solids | Form A |
| 59 | ACN | Thin Slurry | Form C (very small amount) |
| 60 | Toluene | White solids | Form A |
| 61 | EtOAc | White solids | Form A |
| 62 | IPAc | White solids | Form A |
| 63 | 1,4-Dioxane | Thin slurry | Form C (very small amount) |
| 64 | Water | White solids | Form A |
| 65 | CHCl₃ | Gel | Amorphous |

For the liquid vapor diffusion experiments, approximately 30 mg of free base Form A of Compound 1, as discussed in Example 1, was dissolved in different solvents to obtain saturated solutions in 4 mL vials which were placed in 20 mL vials containing anti-solvents. The obtained solids were characterized by XRPD after 12 days. Table 14 discloses the parameters and resulting crystal forms of liquid vapor diffusion experiments.

TABLE 14

| Exp. | | | | Crystal Form |
|---|---|---|---|---|
| ID | Solvent | Anti-solvent | Observation | Obtained |
| 66 | Acetone | MTBE | Glassy material | Amorphous |
| 67 | | Hexane | Glassy material | Amorphous |
| 68 | | H₂O | Glassy material | Amorphous |
| 69 | 2-MeTHF | heptane | White solids | Amorphous |
| 70 | | Toluene | Gel | Amorphous |
| 71 | | MTBE | Clear | N/A |
| 72 | NMP | water | White solids | Form A |
| 73 | | IPA | White solids | Form C (very small |
| | | | | amount) |
| 74 | | MTBE | White solids | Form C (very small |
| | | | | amount) |

For the slow evaporation experiments, evaporation occurred at either room temperature of 50° C. To obtain saturated solutions, approximately 30 mg of free base Form A of Compound 1, as discussed in Example 1, was dissolved in different solvents in 4 mL vials. The vials were covered with paraffin film with 3-5 holes and placed at room temperature or 50° C. for evaporation. The obtained solids were characterized by XRPD. Table 15 discloses the parameters and resulting crystal forms of slow evaporation experiments.

TABLE 15

| Exp. | | Temp. | | Crystal Form |
|---|---|---|---|---|
| ID | Solvent | (° C.) | Observation | Obtained |
| 75 | Acetone | Room | Glassy | Amorphous |
| 76 | THF | Temperature | Glassy | Amorphous |
| 77 | ACN | | White solid | Form C and A (small |
| | | | | amount) |
| 78 | DCM | | Glassy | Amorphous |
| 79 | DMA | | Gel | Amorphous |
| 80 | IPAC | 50 | White solids | Form C (very small |
| | | | | amount) |
| 81 | 2-MeTHF | | White solids | Form C (very small |
| | | | | amount) |
| 82 | MEK | | Glassy | Amorphous |
| 83 | 1,4-Dioxane | | Gel | Amorphous |
| 84 | Acetic acid | | Glassy | Amorphous |

For the slow cooling experiments, approximately 30 mg of free base Form A of Compound 1, as discussed in Example 1, was dissolved in different solvents to obtain saturated solutions in 4 mL vials at 55° C. using a hot plate. The solutions were slowly cooled down to room temperature using the following ramp: 55° C. for 3 hours, 40° C. for 4 hours, 25° C. for 4 hours, 15° C. for 5 hours, and 10° C. for 5 hours. The obtained solids were characterized by XRPD. Table 16 discloses the parameters and resulting crystal forms of slow cooling experiments.

TABLE 16

| Exp. ID | Solvent (v:v) | Observation | Crystal Form Obtained |
|---|---|---|---|
| 85 | ACN | White solids | Form A |
| 86 | 2Me-THF | On-going | Form C (very small amount) |
| 87 | MeOH | White solids | Form C (very small amount) |
| 88 | MIBK | Gel | Amorphous |
| 89 | CHCl3 | Glassy | Amorphous |
| 90 | IPAc | White solids | Form C (very small amount) |
| 91 | EtOH | White solids | Form B |

For the polymer induced crystallization experiments, approximately 30 mg of free base Form A of Compound 1, as discussed in Example 1, was dissolved in different solvents to create saturated solutions. Either polyvinylpyrrolidone (PVP), polyethylene glycol (PEO), polyvinyl alcohol (PVA), or hydroxypropyl methylcellulose (HPMC) were added to the saturated solutions to induce heteronucleation. The obtained solids were characterized by XRPD after 12 days. Table 17 discloses the parameters and resulting crystal forms of polymer induced crystallization experiments.

TABLE 17

| Exp. | | | | Crystal |
|---|---|---|---|---|
| ID | Solvent | Polymer | Observation | Form Obtained |
| 92 | MeOH | PVP | White solid | Form D |
| 93 | DCM | | Glassy | Amorphous |
| 94 | NMP | | Glassy | N/A |
| 95 | MEK | PEO (Poloxamer) | Glassy | Amorphous |
| 96 | 1,4-Dioxane | PVA | Gel | Amorphous |
| 97 | THF | PEO (Poloxamer) | Glassy | Amorphous |
| 98 | MeOH | PVA | White solid | Form D |
| 99 | Acetic acid | PEO (Poloxamer) | Glassy | Amorphous |
| 100 | 2-BuOH | HPMC | White solids | Form B |

### Example 7: Pre-Clinical Bioperformance

The pharmacokinetics of different crystalline forms of Compound 1 was investigated following oral administration of Free Base Form A, HCl Form A, and HCl Form B of Compound 1 in male beagle dogs at a dose of 100 mg/dog (˜Approximately 10 mg per kg). The substances were filled into Empty HPMC Capsules after correcting for potency and administered to dogs pre-treated with either pentagastrin or famotidine along with 40 mL of water. Pentagastrin enhances gastric secretions whereas famotidine neutralizes gastric sections. Therefore, pentagastrin and famotidine pre-treatments were used to simulate the impact of gastric pH on bioavailability and reduce the variability in gastric pH between dogs.

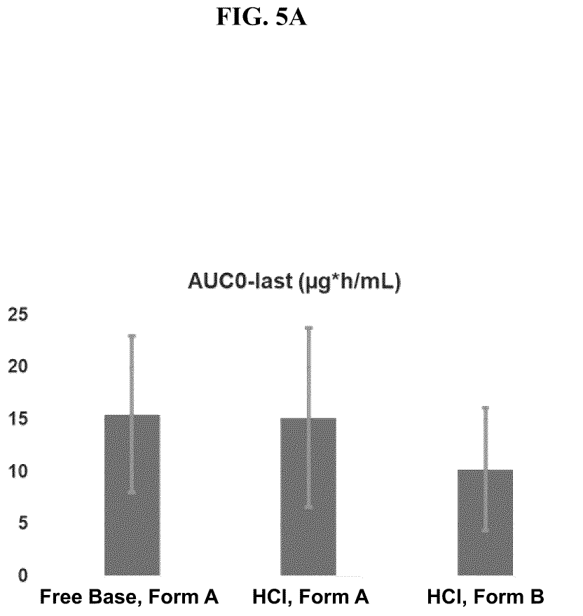

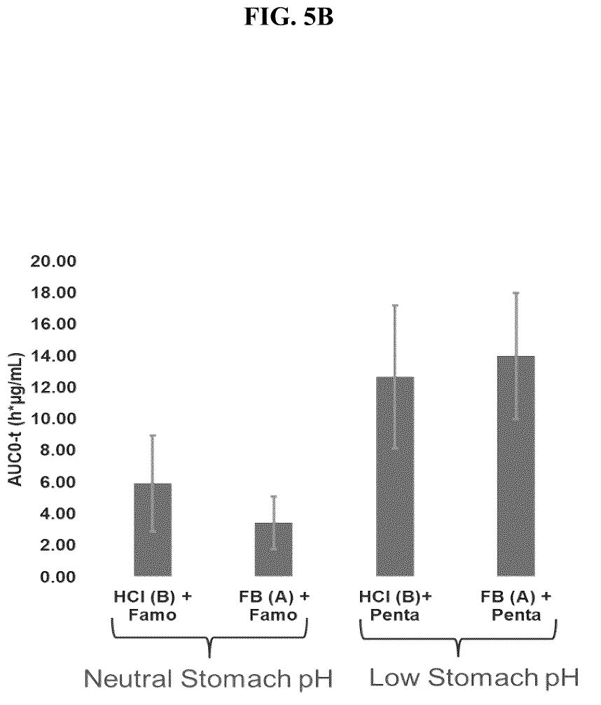

Plasma samples were collected at pre-defined intervals and analyzed for the concentration of Compound 1. The plasma concentration versus time data was analyzed by non-compartmental approaches using Win Nonlin software program to estimate PK parameters such as Cₘₐₓ, Tₘₐₓ, AUC₀₋ₜ and AUC_{0-inf}. These results are shown in FIGS. 5A and 5B.

From the results, it was found that HCl Form A was less stable than HCl Form B and that HCl salt exhibited a common ion effect in the gastric media which may have caused high variability in pentagastrin-treated dogs. Additionally, Free Bass Form A has an equivalent exposure to HCl salt when the particle size was normalized. And additionally, for both HCl forms tested there was marked differences in the bioavailability between dogs treated with pentagastrin and famotidine, which suggests a possible food effect for both forms.

### Example 8: Excipient Compatibility

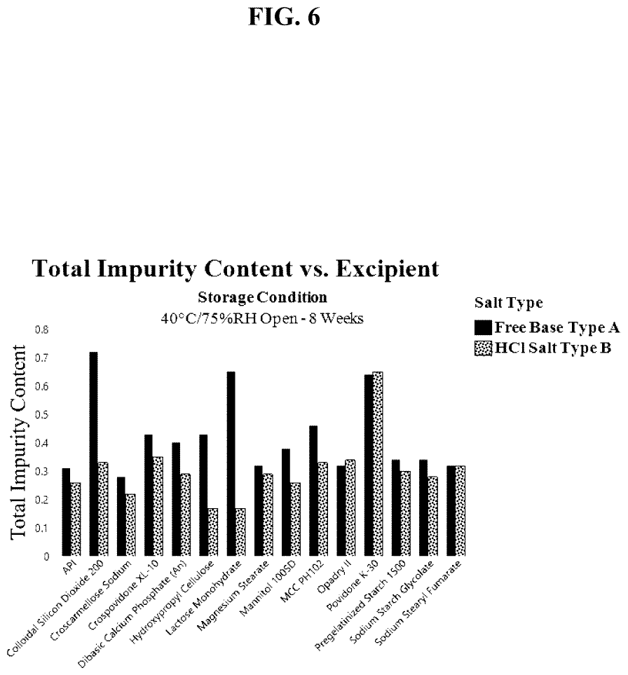

The compatibility of both the Free Base Form A and HCl Form B of Compound 1 with multiple excipients commonly used in Oral Solid Dosage (OSD) formulation development was evaluated. Binary mixtures of samples were created by mixing the substance and the excipients (1:10 Compound to Excipients for fillers and 1:1 for all other excipients) using a mortar and pestle and the resultant mixture was accurately weighed and transferred into sample vials. The excipients used were as follows: colloidal silicon dioxide 200, croscarmellose sodium, crosprovidone XL-10, dibasic calcium phosphate (an), hydroxypropyl cellulose, lactose monohydrate, magnesium stearate, mannitol 100SD, MCC PH102, Opadry™ 11, povidone K-30, pregelatinized starch 1500, sodium starch glycolate, and sodium stearyl fumarate. The sample vials were set down at 40° C./75% RH condition for 8 weeks in open condition before analyzing their impurity levels using HPLC. The results of the total impurity content for these samples are shown in FIG. 6 and are compared to Free Base Form A and HCl Form B of Compound 1 without any excipients.

From FIG. 6, it can be seen that both Free Base Form A and HCl Form B of Compound 1 exhibit good compatibility with excipients. Additionally, Free Base Form A was more sensitive to colloidal silicon dioxide and lactose monohydrate than HCl Form B. Although the risk of chemical stability is slightly higher for the Free Base Form A and HCl Form B, it is still manageable by influencing the choice of excipients selected.

### Example 9: Manufacturability and Physical Stability

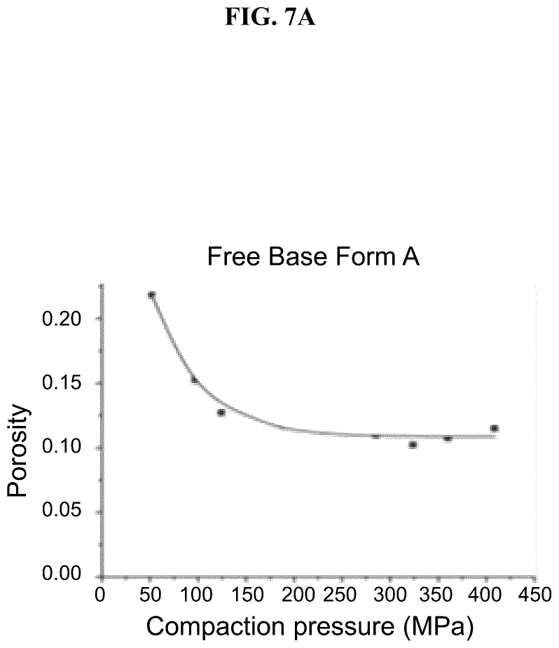

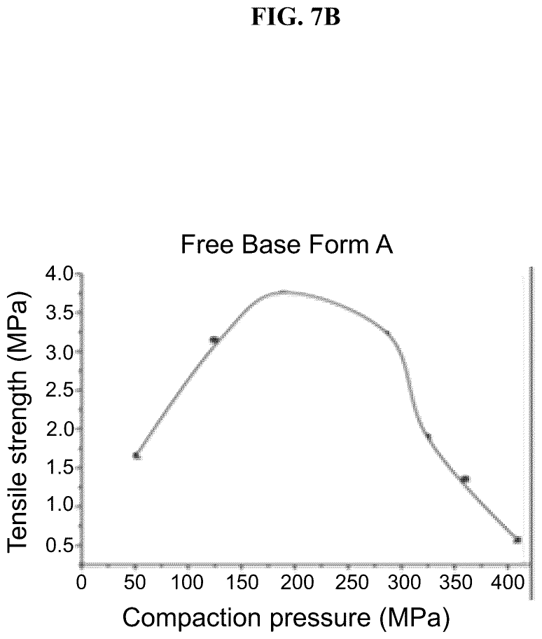

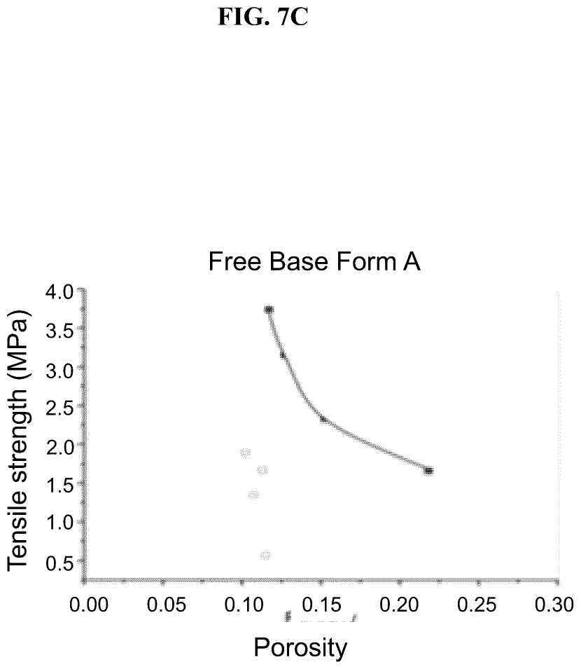

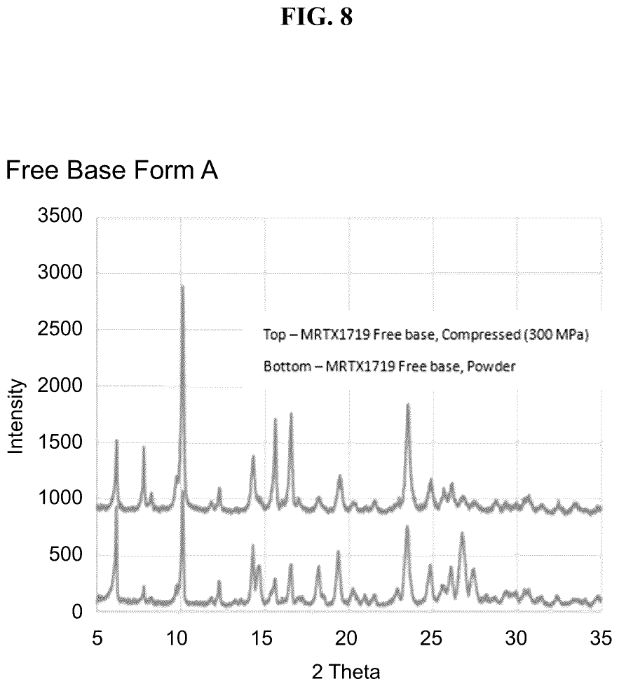

The compressibility (i.e., porosity vs compaction pressure), tabletability (i.e., tensile strength vs compaction pressure), and compatibility (i.e. tensile strength vs porosity) of Free Base Form A of Compound 1 was measured. The results of these tests are shown in FIGS. 7A-7C. Free Base Form A exhibited good compressibility, tabletability, and compatibility. No apparent loss of crystallinity was observed for free base at high compaction pressures (FIG. 8).

### Example 10: Intrinsic Dissolution Rate (IDR) and Drug in Capsule Dissolution in Fasted Gastric Simulated Fluid (FaSSGF) and Fasted Simulated Intestinal Fluid (FaSSIF)

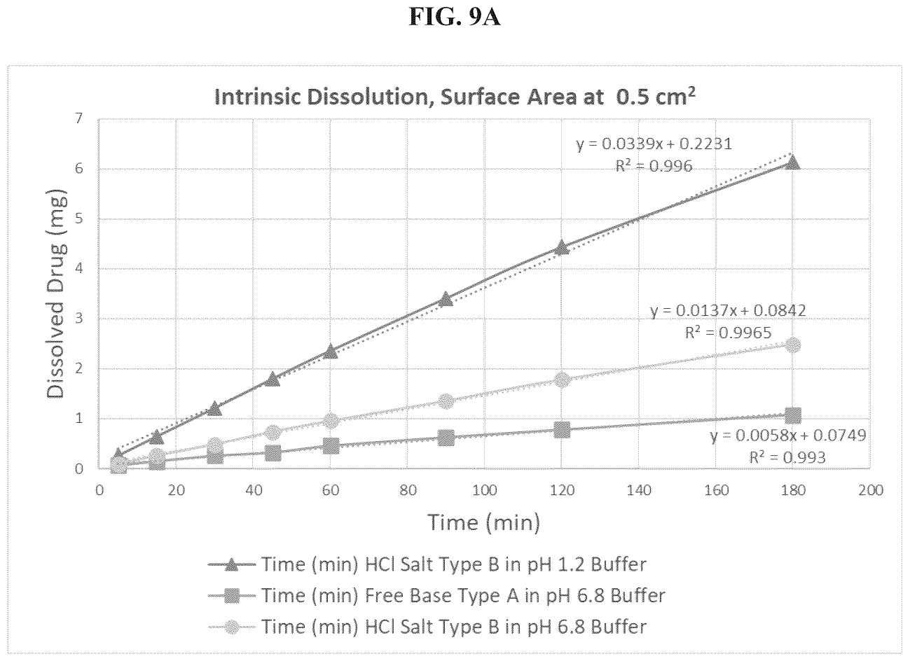

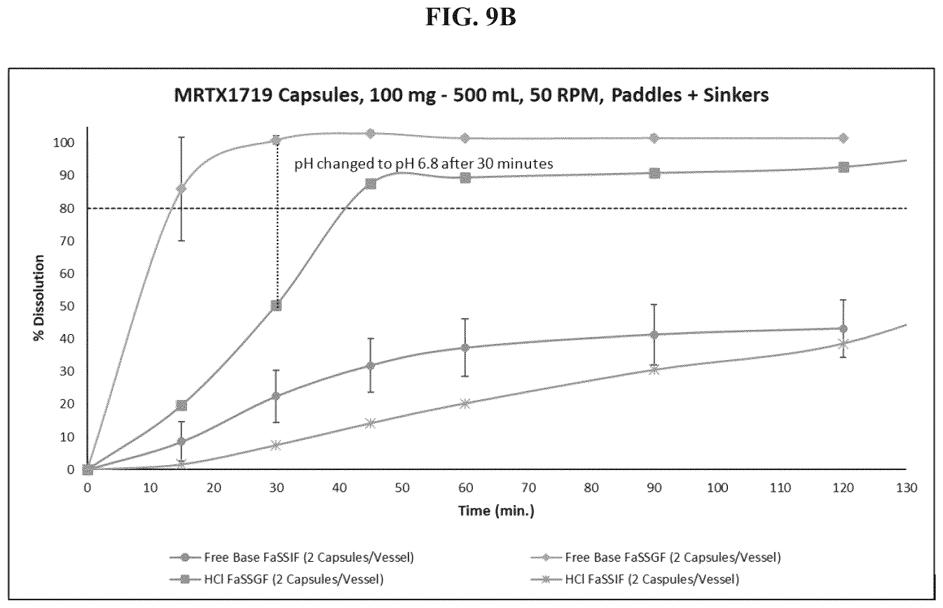

The intrinsic dissolution rate (IDR) was determined by measuring the amount of dissolved Free Base Form A and HCl Form B of Compound 1 in the buffer and then a linear curve of dissolved compounds versus time was constructed. The IDR rate was calculated using the slope of the linear curve divided by the surface area of tablet (0.5 cm²). Free Base Form A and HCl Form B of Compound 1 were pressed into a tablet with 0.8 cm diameter at 14 MPa, respectively. Capsules of HCl Form B were stirred in pH 1.2 and pH 6.8 buffer at 37° C. with 50 rpm in USP Type II dissolution apparatus with sinkers, while tablets of Free Base Form A were stirred in pH 6.8 buffer under the same conditions. 0.6 mL liquid was removed with a syringe at each time point and filtered. The concentration of filtrates was analyzed by HPLC. The IDR results are shown in FIG. 9A and the dissolution in capsules is shown in FIG. 9B.

The Free Base Form A exhibited faster dissolution in gastric media due to common ion effect slowing dissolution of HCl Form B. In intestinal media, HCl Form B has slightly faster dissolution rate than Free Base Form A, however it is observed that Free Base Form A is able to maintain super saturation in intestinal fluid when the pH is shifted from gastric to intestinal after 30 minutes.

### Itemized List of Embodiments

Embodiment 1. A crystalline form of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile free base having an endothermic differential scanning calorimetric (DSC) peak temperature within ±2% of 86° C.

Embodiment 2. The crystalline form of embodiment 1, wherein the endothermic DSC peak temperature is within ±1% of 86° C.

Embodiment 3. The crystalline form of embodiment 1, wherein the endothermic DSC peak temperature is within ±0.5% of 86° C.

Embodiment 4. The crystalline form of embodiment 1, wherein the crystalline form has an endothermic DSC peak temperature within ±2% of 154° C.

Embodiment 5. The crystalline form of embodiment 4, wherein the endothermic DSC peak temperature is within ±1% of 154° C.

Embodiment 6. The crystalline form of embodiment 4, wherein the endothermic DSC peak temperature is within ±0.5% of 154° C.

Embodiment 7. The crystalline form of embodiment 1, wherein the crystalline form has an exothermic DSC peak temperature within ±2% of 166° C.

Embodiment 8. The crystalline form of embodiment 7, wherein the exothermic DSC peak temperature is within ±1% of 166° C.

Embodiment 9. The crystalline form of embodiment 7, wherein the exothermic DSC peak temperature is within ±0.5% of 166° C.

Embodiment 10. The crystalline form of embodiment 1, wherein the crystalline form has an endothermic DSC peak temperature within ±2% of 261° C.

Embodiment 11. The crystalline form of embodiment 10, wherein the endothermic DSC peak temperature is within ±1% of 261° C.

Embodiment 12. The crystalline form of embodiment 10, wherein the endothermic DSC peak temperature is within ±0.5% of 261° C.

Embodiment 13. The crystalline form of embodiment 1, wherein the crystalline form has an exothermic DSC peak temperature within ±2% of 270° C.

Embodiment 14. The crystalline form of embodiment 13, wherein the exothermic DSC peak temperature is within ±1% of 270° C.

Embodiment 15. The crystalline form of embodiment 13, wherein the exothermic DSC peak temperature is within ±0.5% of 270° C.

Embodiment 16. A crystalline form of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile free base having an X-ray powder diffraction (XRPD) pattern comprising a peak at a two-theta angle of 3.1°±0.2°.

Embodiment 17. The crystalline form of embodiment 16, wherein the crystalline form has an X-ray powder diffraction (XRPD) pattern comprising a peak at a two-theta angle of 6.2°±0.2°.

Embodiment 18. The crystalline form of embodiment 16, wherein the crystalline form has an X-ray powder diffraction (XRPD) pattern comprising a peak at a two-theta angle of 10.2°±0.2°.

Embodiment 19. The crystalline form of embodiment 16, wherein the crystalline form has an X-ray powder diffraction (XRPD) pattern comprising a peak at a two-theta angle of 14.3°±0.2°.

Embodiment 20. The crystalline form of embodiment 16, wherein the crystalline form has an X-ray powder diffraction (XRPD) pattern comprising peaks at a two-theta angles of 3.1°±0.2°, 6.2°±0.2°, 10.2°±0.2°, and 14.3°±0.2°.

Embodiment 21. A crystalline form of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile free base having an X-ray powder diffraction pattern substantially shown in FIG. 1B.

Embodiment 22. The crystalline form of any one of embodiments 1-21, wherein the crystalline form is a sesquihydrate.

Embodiment 23. The crystalline form of any one of embodiments 1-22, wherein the crystalline form has a thermal gravimetric analysis (TGA) plot comprising a mass loss of about 4.4% when heated from about 25° C. to about 150° C.

Embodiment 24. The crystalline form of any one of embodiments 1-23, wherein the crystalline form has a purity of at least 97% by weight of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile free base.

Embodiment 25. The crystalline form of any one of embodiments 1-24, wherein the crystalline form has a purity of at least 98% by weight of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile free base.

Embodiment 26. A pharmaceutical composition comprising the crystalline form of any one of embodiments 1-25 and a pharmaceutically acceptable carrier.

Embodiment 27. A crystalline form of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile free base having an endothermic differential scanning calorimetric (DSC) peak temperature within ±2% of 55° C.

Embodiment 28. The crystalline form of embodiment 27, wherein the endothermic DSC peak temperature is within ±1% of 55° C.

Embodiment 29. The crystalline form of embodiment 27, wherein the endothermic DSC peak temperature is within ±0.5% of 55° C.

Embodiment 30. The crystalline form of embodiment 27, wherein the crystalline form has an endothermic DSC peak temperature within ±2% of 144° C.

Embodiment 31. The crystalline form of embodiment 30, wherein the endothermic DSC peak temperature is within ±1% of 144° C.

Embodiment 32. The crystalline form of embodiment 30, wherein the endothermic DSC peak temperature is within ±0.5% of 144° C.

Embodiment 33. The crystalline form of embodiment 27, wherein the crystalline form has an exothermic DSC peak temperature within ±2% of 168° C.

Embodiment 34. The crystalline form of embodiment 33, wherein the exothermic DSC peak temperature is within ±1% of 168° C.

Embodiment 35. The crystalline form of embodiment 33, wherein the exothermic DSC peak temperature is within ±0.5% of 168° C.

Embodiment 36. The crystalline form of embodiment 27, wherein the crystalline form has an endothermic DSC peak temperature within ±2% of 260° C.

Embodiment 37. The crystalline form of embodiment 36, wherein the endothermic DSC peak temperature is within ±1% of 260° C.

Embodiment 38. The crystalline form of embodiment 36, wherein the endothermic DSC peak temperature is within ±0.5% of 260° C.

Embodiment 39. The crystalline form of embodiment 27, wherein the crystalline form has an exothermic DSC peak temperature within ±2% of 268° C.

Embodiment 40. The crystalline form of embodiment 39, wherein the exothermic DSC peak temperature is within ±1% of 268° C.

Embodiment 41. The crystalline form of embodiment 39, wherein the exothermic DSC peak temperature is within ±0.5% of 268° C.

Embodiment 42. A crystalline form of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile free base having an X-ray powder diffraction (XRPD) pattern comprising a peak at a two-theta angle of 6.4°±0.2°.

Embodiment 43. The crystalline form of embodiment 42, wherein the crystalline form has an X-ray powder diffraction (XRPD) pattern comprising a peak at a two-theta angle of 12.2°+0.2°.

Embodiment 44. The crystalline form of embodiment 42, wherein the crystalline form has an X-ray powder diffraction (XRPD) pattern comprising a peak at a two-theta angle of 12.8°±0.2°.

Embodiment 45. The crystalline form of embodiment 42, wherein the crystalline form has an X-ray powder diffraction (XRPD) pattern comprising a peak at a two-theta angle of 24.5°±0.2°.

Embodiment 46. The crystalline form of embodiment 42, wherein the crystalline form has an X-ray powder diffraction (XRPD) pattern comprising a peak at a two-theta angle of 25.0°±0.2°.

Embodiment 47. The crystalline form of embodiment 42, wherein the crystalline form has an X-ray powder diffraction (XRPD) pattern comprising peaks at a two-theta angle of 6.4°±0.2°, 12.2°±0.2°, 12.8°±0.2°, 24.5°±0.2°, and 25.0°±0.2°.

Embodiment 48. A crystalline form of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile free base having an X-ray powder diffraction pattern substantially shown in FIG. 2B.

Embodiment 49. The crystalline form of any one of embodiments 27-48, wherein the crystalline form is a dihydrate.

Embodiment 50. The crystalline form of any one of embodiments 27-49, wherein the crystalline form has a thermal gravimetric analysis (TGA) plot comprising a mass loss of about 6.5% when heated from about 25° C. to about 150° C.

Embodiment 51. The crystalline form of any one of embodiments 27-50, wherein the crystalline form has a purity of at least 97% by weight of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile free base.

Embodiment 52. The crystalline form of any one of embodiments 27-51, wherein the crystalline form has a purity of at least 98% by weight of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile free base.

Embodiment 53. A pharmaceutical composition comprising the crystalline form of any one of embodiments 27-52 and a pharmaceutically acceptable carrier.

Embodiment 54. A crystalline form of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile free base having an endothermic differential scanning calorimetric (DSC) peak temperature within ±2% of 28° C.

Embodiment 55. The crystalline form of embodiment 54, wherein the endothermic DSC peak temperature is within ±1% of 28° C.

Embodiment 56. The crystalline form of embodiment 54, wherein the endothermic DSC peak temperature is within ±0.5% of 28° C.

Embodiment 57. The crystalline form of embodiment 54, wherein the crystalline form has an endothermic DSC peak temperature within ±2% of 100° C.

Embodiment 58. The crystalline form of embodiment 57, wherein the endothermic DSC peak temperature is within ±1% of 100° C.

Embodiment 59. The crystalline form of embodiment 57, wherein the endothermic DSC peak temperature is within ±0.5% of 100° C.

Embodiment 60. The crystalline form of embodiment 54, wherein the crystalline form has an exothermic DSC peak temperature within ±2% of 161° C.

Embodiment 61. The crystalline form of embodiment 60, wherein the exothermic DSC peak temperature is within ±1% of 161° C.

Embodiment 62. The crystalline form of embodiment 60, wherein the exothermic DSC peak temperature is within ±0.5% of 161° C.

Embodiment 63. The crystalline form of embodiment 54, wherein the crystalline form has an endothermic DSC peak temperature within ±2% of 269° C.

Embodiment 64. The crystalline form of embodiment 63, wherein the endothermic DSC peak temperature is within ±1% of 269° C.

Embodiment 65. The crystalline form of embodiment 63, wherein the endothermic DSC peak temperature is within ±0.5% of 269° C.

Embodiment 66. The crystalline form of embodiment 54, wherein the crystalline form has an exothermic DSC peak temperature within ±2% of 276° C.

Embodiment 67. The crystalline form of embodiment 66, wherein the exothermic DSC peak temperature is within ±1% of 276° C.

Embodiment 68. The crystalline form of embodiment 66, wherein the exothermic DSC peak temperature is within ±0.5% of 276° C.

Embodiment 69. A crystalline form of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile free base having an X-ray powder diffraction (XRPD) pattern comprising a peak at a two-theta angle of 8.2°±0.2°.

70. The crystalline form of embodiment 69, wherein the crystalline form has an X-ray powder diffraction (XRPD) pattern comprising a peak at a two-theta angle of 9.7°±0.2°.

Embodiment 71. The crystalline form of embodiment 69, wherein the crystalline form has an X-ray powder diffraction (XRPD) pattern comprising a peak at a two-theta angle of 15.1°±0.2°.

Embodiment 72. The crystalline form of embodiment 69, wherein the crystalline form has an X-ray powder diffraction (XRPD) pattern comprising a peak at a two-theta angle of 24.8°±0.2°.

Embodiment 73. The crystalline form of embodiment 69, wherein the crystalline form has an X-ray powder diffraction (XRPD) pattern comprising peaks at a two-theta angle of 8.2°±0.2°, 9.7°±0.2°, 15.1°±0.2°, and 24.8°±0.2°.

Embodiment 74. A crystalline form of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile free base having an X-ray powder diffraction pattern substantially shown in FIG. 3B.

Embodiment 75. The crystalline form of any one of embodiments 54-74, wherein the crystalline form is a channel hydrate (e.g., solvate, racemate).

Embodiment 76. The crystalline form of any one of embodiments 54-75, wherein the crystalline form has a thermal gravimetric analysis (TGA) plot comprising a mass loss of about 3.3% when heated from about 25° C. to about 200° C.

Embodiment 77. The crystalline form of any one of embodiments 54-76, wherein the crystalline form has a purity of at least 97% by weight of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile free base.

Embodiment 78. The crystalline form of any one of embodiments 54-77, wherein the crystalline form has a purity of at least 98% by weight of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile free base.

Embodiment 79. A pharmaceutical composition comprising the crystalline form of any one of embodiments 54-78 and a pharmaceutically acceptable carrier.

Embodiment 80. A crystalline form of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile free base having an endothermic differential scanning calorimetric (DSC) peak temperature within ±2% of 59° C.

Embodiment 81. The crystalline form of embodiment 80, wherein the endothermic DSC peak temperature is within ±1% of 59° C.

Embodiment 82. The crystalline form of embodiment 80, wherein the endothermic DSC peak temperature is within ±0.5% of 59° C.

Embodiment 83. The crystalline form of embodiment 80, wherein the endothermic crystalline form has an endothermic DSC peak temperature within ±2% of 140° C.

Embodiment 84. The crystalline form of embodiment 83, wherein the endothermic DSC peak temperature is within ±1% of 140° C.

Embodiment 85. The crystalline form of embodiment 83, wherein the endothermic DSC peak temperature is within ±0.5% of 140° C.

Embodiment 86. The crystalline form of embodiment 80, wherein the crystalline form has an exothermic DSC peak temperature within ±2% of 161° C.

Embodiment 87. The crystalline form of embodiment 86, wherein the exothermic DSC peak temperature is within ±1% of 161° C.

Embodiment 88. The crystalline form of embodiment 86, wherein the exothermic DSC peak temperature is within ±0.5% of 161° C.

Embodiment 89. The crystalline form of embodiment 80, wherein the crystalline form has an endothermic DSC peak temperature within ±2% of 258° C.

Embodiment 90. The crystalline form of embodiment 89, wherein the endothermic DSC peak temperature is within ±1% of 258° C.

Embodiment 91. The crystalline form of embodiment 89, wherein the endothermic DSC peak temperature is within ±0.5% of 258° C.

Embodiment 92. The crystalline form of embodiment 80, wherein the crystalline form has an exothermic DSC peak temperature within ±2% of 267° C.

Embodiment 93. The crystalline form of embodiment 92, wherein the exothermic DSC peak temperature is within ±1% of 267° C.

Embodiment 94. The crystalline form of embodiment 92, wherein the exothermic DSC peak temperature is within ±0.5% of 267° C.

Embodiment 95. A crystalline form of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile free base having an X-ray powder diffraction (XRPD) pattern comprising a peak at a two-theta angle of 5.4°±0.2°.

Embodiment 96. The crystalline form of embodiment 95, wherein the crystalline form has an X-ray powder diffraction (XRPD) pattern comprising a peak at a two-theta angle of 9.9°±0.2°.

Embodiment 97. The crystalline form of embodiment 95, wherein the crystalline form has an X-ray powder diffraction (XRPD) pattern comprising a peak at a two-theta angle of 13.7°±0.2°.

Embodiment 98. The crystalline form of embodiment 95, wherein the crystalline form has an X-ray powder diffraction (XRPD) pattern comprising a peak at a two-theta angle of 27.1°±0.2°.

Embodiment 99. The crystalline form of embodiment 95, wherein the crystalline form has an X-ray powder diffraction (XRPD) pattern comprising peaks at a two-theta angle of 5.4°±0.2°, 9.9°±0.2°, 13.7°±0.2°, and 27.1°±0.2°.

Embodiment 100. A crystalline form of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile free base having an X-ray powder diffraction pattern substantially shown in FIG. 4B.

Embodiment 101. The crystalline form of any one of embodiments 80-100, wherein the crystalline form is a hydrate.

Embodiment 102. The crystalline form of any one of embodiment 80-101, wherein the crystalline form has a thermal gravimetric analysis (TGA) plot comprising a mass loss of about 6.9% when heated from about 25° C. to about 150° C.

Embodiment 103. The crystalline form of any one of embodiments 80-102, wherein the crystalline form has a purity of at least 97% by weight of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile free base.

Embodiment 104. The crystalline form of any one of embodiments 80-103, wherein the crystalline form has a purity of at least 98% by weight of 2-(4-(4-(aminomethyl)-1-oxo-1,2-dihydrophthalazin-6-yl)-1-methyl-1h-pyrazol-5-yl)-4-chloro-6-cyclopropoxy-3-fluorobenzonitrile free base.

Embodiment 105. A pharmaceutical composition comprising the crystalline form of any one of embodiments 80-104 and a pharmaceutically acceptable carrier.

Embodiment 106. A method for treating cancer in a subject in need thereof, comprising administering to the subject a crystalline form of a crystalline form of any one of embodiments 1-25, 27-52, 54-78, or 80-104, or a pharmaceutical composition comprising a crystalline form of any one of embodiments 26,53, 79 or 105.

Embodiment 107. A method according to Embodiment 106 wherein the cancer is a MTAP-associated cancer.

Embodiment 108. A method according to Embodiment 106 wherein the cancer is selected from the group consisting of ovarian serous cystadenocarcinoma, squamous cell lung cancer, lung adenocarcinoma, mesothelioma; esophogeal squamous cell carcinoma, gastric adenocarcinoma, pancreatic ductal adenocarcinoma, kidney adenocarcinoma, bladder transitional cell carcinoma, hepatocellular carcinoma, cholangiocarcinoma, cholangiocarcinoma; osteosarcoma, multiple myeloma, astrocytoma, glioma, glioblastoma, uterine sarcoma, acute myeloid leukemia, acute lymphoblastic leukemia, non-Hodgkin's lymphoma, malignant melanoma, endometrial carcinoma and thyroid carcinoma.

Embodiment 109. A method according to Embodiment 106 wherein the cancer is selected from the group consisting of bladder cancer, bone cancer, brain cancer, blood cancer, breast cancer, colorectal cancer, esophageal cancer, gastric cancer, head and neck cancer, kidney cancer, liver cancer, lung cancer, mesothelioma, ovarian cancer, pancreatic cancer, skin cancer, thyroid cancer and uterine cancer

While the invention has been described in connection with specific embodiments thereof, it will be understood that it is capable of further modifications and this application is intended to cover any variations, uses, or adaptations of the invention following, in general, the principles of the invention and including such departures from this disclosure as come within known or customary practice within the art to which the invention pertains and as may be applied to the essential features hereinbefore set forth, and as follows in the scope of the appended claims.

What is claimed is:

1. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile free base having an endothermic differential scanning calorimetric (DSC) peak temperature within ±2% of 86° C.

2. The crystalline form of claim 1, wherein the crystalline form has an endothermic DSC peak temperature within ±2% of 154° C.

3. The crystalline form of claim 1, wherein the crystalline form has an exothermic DSC peak temperature within ±2% of 166° C.

4. The crystalline form of claim 1, wherein the crystalline form has an endothermic DSC peak temperature within ±2% of 261° C.

5. The crystalline form of claim 1, wherein the crystalline form has an exothermic DSC peak temperature within ±2% of 270° C.

6. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile free base having an X-ray powder diffraction (XRPD) pattern comprising a peak at a two-theta angle of 3.1°±0.2°.

7. The crystalline form of claim 6, wherein the crystalline form has an X-ray powder diffraction (XRPD) pattern comprising peaks at a two-theta angles of 3.1°±0.2°, 6.2°±0.2°, 10.2°±0.2°, and 14.3°±0.2°.

8. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile free base having an X-ray powder diffraction pattern substantially as shown in FIG. 1B.

9. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile free base having an endothermic differential scanning calorimetric (DSC) peak temperature within ±2% of 55° C.

10. The crystalline form of claim 9, wherein the crystalline form has an endothermic DSC peak temperature within ±2% of 144° C.

11. The crystalline form of claim 9, wherein the crystalline form has an exothermic DSC peak temperature within ±2% of 168° C.

12. The crystalline form of claim 9, wherein the crystalline form has an endothermic DSC peak temperature within ±2% of 260° C.

13. The crystalline form of claim 9, wherein the crystalline form has an exothermic DSC peak temperature within ±2% of 268° C.

14. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile free base having an X-ray powder diffraction (XRPD) pattern comprising a peak at a two-theta angle of 6.4°±0.2°.

15. The crystalline form of claim 14, wherein the crystalline form has an X-ray powder diffraction (XRPD) pattern comprising peaks at a two-theta angle of 6.4°±0.2°, 12.2°±0.2°, 12.8°±0.2°, 24.5°±0.2, and 25.0°±0.2°.

16. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile free base having an X-ray powder diffraction pattern substantially as shown in FIG. 2B.

17. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile free base having an endothermic differential scanning calorimetric (DSC) peak temperature within ±2% of 28° C.

18. The crystalline form of claim 17, wherein the crystalline form has an endothermic DSC peak temperature within ±2% of 100° C.

19. The crystalline form of claim 17, wherein the crystalline form has an exothermic DSC peak temperature within ±2% of 161° C.

20. The crystalline form of claim 17, wherein the crystalline form has an endothermic DSC peak temperature within ±2% of 269° C.

21. The crystalline form of claim 17, wherein the crystalline form has an exothermic DSC peak temperature within ±2% of 276° C.

22. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile free base having an X-ray powder diffraction (XRPD) pattern comprising a peak at a two-theta angle of 8.2°±0.2°.

23. The crystalline form of claim 22, wherein the crystalline form has an X-ray powder diffraction (XRPD) pattern comprising peaks at a two-theta angle of 8.2°±0.2°, 9.7°±0.2°, 15.1°±0.2°, and 24.8°±0.2°.

24. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile free base having an X-ray powder diffraction pattern substantially as shown in FIG. 3B.

25. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile free base having an endothermic differential scanning calorimetric (DSC) peak temperature within ±2% of 59° C.

26. The crystalline form of claim 25, wherein the endothermic crystalline form has an endothermic DSC peak temperature within ±2% of 140° C.

27. The crystalline form of claim 25, wherein the crystalline form has an exothermic DSC peak temperature within ±2% of 161° C.

28. The crystalline form of claim 25, wherein the crystalline form has an endothermic DSC peak temperature within ±2% of 258° C.

29. The crystalline form of claim 25, wherein the crystalline form has an exothermic DSC peak temperature within ±2% of 267° C.

30. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile free base having an X-ray powder diffraction (XRPD) pattern comprising a peak at a two-theta angle of 5.4°±0.2°.

31. The crystalline form of claim 30, wherein the crystalline form has an X-ray powder diffraction (XRPD) pattern comprising peaks at a two-theta angle of 5.4°±0.2°, 9.9°±0.2°, 13.7°±0.2°, and 27.1°±0.2°.

32. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile free base having an X-ray powder diffraction pattern substantially as shown in FIG. 4B.
